(19) United States
(12) Patent Application Publication
(10) Pub. No.: US 2024/0150353 A1
(43) Pub. Date: May 9, 2024

(54) 2-SUBSTITUTED BICYCLO[1.1.1]PENTANES

(71) Applicant: The Trustees of Princeton University, Princeton, NJ (US)

(72) Inventors: David W. MacMillan, Princeton, NJ (US); Olivia L. Garry, Princeton, NJ (US); Yufan Liang, Cambridge, MA (US); Michael Heilmann, Princeton, NJ (US)

(21) Appl. No.: 18/547,462

(22) PCT Filed: Feb. 22, 2022
(86) PCT No.: PCT/US2022/070770

§ 371 Date: Aug. 22, 2023

(51) Int. Cl. C07D 471/04; C07C 51/363; C07C 67/08; C07C 69/753; C07C 69/757; C07C 69/76; C07D 209/08; C07D 209/48; C07D 209/88; C07D 213/26; C07D 213/55; C07D 231/16; C07D 231/56; C07D 403/08; C07D 487/04

(52) U.S. Cl. CPC C07D 471/04; C07C 51/363; C07C 67/08; C07C 69/753; C07C 69/757; C07C 69/76; C07D 209/08; C07D 209/48; C07D 209/88; C07D 213/26; C07D 213/55; C07D 231/16; C07D 231/56; C07D 403/08; C07D 487/04; C07C 2602/38

(57) ABSTRACT

Provided herein are 2-substituted bicyclo[1.1.1]pentane (BCP) compounds, as well as methods of making the 2-substituted BCP compounds and methods of derivatizing the 2-substituted BCP compounds, particularly at the 2-position. The 2-substituted BCP compounds described herein are useful building blocks in the synthesis of a variety of products, including pharmaceuticals, polymers, liquid crystals, monolayers and supramolecular structures.

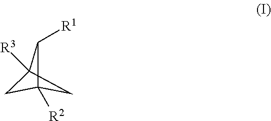

### RELATED APPLICATION

This application claims the benefit of U.S. Provisional Application No. 63/151,974, filed on Feb. 22, 2021. The entire teachings of this application are incorporated herein by reference.

### GOVERNMENT SUPPORT

This invention was made with government support under Grant No. GM134897 awarded by the National Institutes of Health. The government has certain rights in the invention.

### BACKGROUND

Bicyclo[1.1.1]pentane (BCP) derivatives are of interest in a variety of settings, from materials science to pharmaceuticals. BCP derivatives are valued in materials science for their rigidity, lack of chemical reactivity, transparency and thermal stability, and have been incorporated into polymers, liquid crystals, monolayers and supramolecular structures, such as molecular rods and rotors. In the pharmaceutical industry, BCP derivatives have been used instead of small aromatic rings, such as mono- and para-substituted phenyls, and tent-butyl and alkynyl groups to increase the solubility, metabolic stability, permeability and partition coefficient of pharmaceuticals. Successful replacement of phenyl with 1-substituted or 1,3-disubstituted BCP in pharmaceuticals has heretofore been most successful when the phenyl being replaced by the BCP serves the role of a spacer, and does not participate in pi interactions with its target, which 1-substituted and 1,3-disubstituted BCPs cannot provide.

1-Substituted BCPs and 1,3-disubstituted BCPs are well-studied, and synthetic routes to produce them can be found in the literature. 2-Monosubstituted, 1,2-disubstituted and 1,2,3-trisubstituted BCPs are largely inaccessible by reported methods.

Accordingly, there is a need for methods of making 2-substituted BCPs, for example, for use as building blocks in the construction of larger molecules.

### SUMMARY

Provided herein are 2-substituted BCPs (e.g., 2-monosubstituted, 1,2-disubstituted, 1,2,3-trisubstituted BCPs), as well as methods of making the 2-substituted BCPs and methods of derivatizing the 2-substituted BCPs, particularly at the 2-position. The 2-substituted BCPs described herein can, by derivatization, for example, be incorporated into a variety of products, including pharmaceuticals, polymers, liquid crystals, monolayers and supramolecular structures, for example, to impart desirable characteristic(s) to the product.

One embodiment is a compound of the following structural formula:

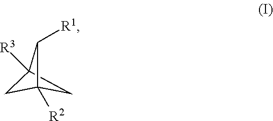

or a salt thereof, wherein values for the variables (e.g., R¹, R², R³) are as described herein.

Another embodiment is a method of making a compound of structural formula (I), or a salt thereof, comprising reacting a compound of the following structural formula:

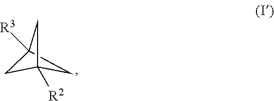

or a salt thereof, with a hydrogen atom abstractor and an le source, thereby making the compound of structural formula I, or a salt thereof. Values for the variables in structural formulas I and I′ (e.g., R¹, R², R³) are as described herein.

Yet another embodiment is a method of derivatizing a compound of structural formula (I), or a salt thereof, the method comprising subjecting the compound of structural formula I, or suitably protected form thereof, or a salt of either of the foregoing, to a derivatization reaction, thereby derivatizing the compound of structural formula I, or a salt thereof. Values for the variables in structural formula I (e.g., R¹, R², R³) are as described herein.

Yet another embodiment is a method of making a compound of structural formula (IV):

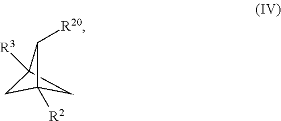

or a salt thereof, comprising reacting a compound of structural formula (I):

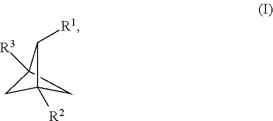

or a salt thereof, with an R¹ activator, a photocatalyst and R²⁰-M in a solvent, thereby making the compound of structural formula (IV), or a salt thereof. Values for the variables (e.g., R¹, R², R³, R²⁰, M) are as described herein.

As building blocks in the construction of larger molecules, the 2-substituted BCPs described herein provide access to a variety of previously-inaccessible molecules, such as 2-substituted BCP-containing pharmaceuticals wherein the BCP replaces a phenyl in a parent compound.

### DETAILED DESCRIPTION

A description of example embodiments follows.

### Definitions

As used herein, the following definitions shall apply unless otherwise indicated.

For purposes of this invention, the chemical elements are identified in accordance with the Periodic Table of the Elements, CRC Handbook of Chemistry and Physics, 100^{th} Ed. General principles of organic chemistry are described in Sorrell, T. Organic Chemistry, 2^{nd} Ed., Sausalito, University Science Books, 2005; and Smith, M. B. March's Advanced Organic Chemistry: Reactions, Mechanisms, and Structure, 7^{th} Ed., New York, J. John Wiley & Sons, 2001, the entire contents of which are hereby incorporated by reference. Unless specified otherwise, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

The terms “a,” “an,” “the” and similar terms used in the context of the present disclosure (especially in the context of the claims) are to be construed to cover both the singular and plural unless otherwise indicated or clearly contradicted by the context. Further, each member of the plural can be the same as or different from the other members.

The term “about,” when referring to a measurable value such as an amount, a temporal duration, and the like, refers to variations of ±20% or, in some instances, ±10% or, in some instances, ±5% or, in some instances, ±1% or, in some instances, ±0.1% from the specified value, as such variations are appropriate to perform the present inventions.

“Bicyclo[1.1.1]pentane” and “BCP” refer to a compound having the following structural formula, wherein the numbers adjacent the carbon atoms in the structural formula refer to the carbon numbers for IUPAC naming purposes:

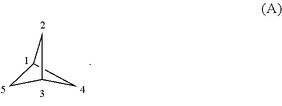

Carbons 1 and 3 in structural formula A are also referred to as bridgehead carbons, and carbons 2, 4 and 5 are also referred to as bridging carbons. Unless the context clearly indicates otherwise, “bicyclo[1.1.1]pentane” and “BCP” are also used herein to refer to derivatives of BCP, such as the BCPs (e.g., 2-substituted BCPs) represented by structural formulas I, I′, II, III and III′, and their salts. Used in the more generic sense to refer to a derivative of BCP, the term is typically preceded by “a,” “an” or “the,” or a similar term, and/or modified with a phrase indicating functionalization or derivatization of the core structure of BCP, such as in the phrase “2-substituted BCP.” When a particular “position” of a BCP is described herein by reference to a numeral, the position denoted by the numeral corresponds to the carbon number having the same numeral in structural formula A. Thus, the “2-position” of a BCP corresponds to 2-carbon in structural formula A.

“Aliphatic,” as used herein, refers to a non-aromatic, branched, straight-chain or cyclic, hydrocarbon radical having the specified number of carbon atoms. Thus, “(C₁-C₁₀)aliphatic” refers to an aliphatic radical having from one to 10 carbon atoms. In some embodiments, aliphatic is (C₁-C₂₅)aliphatic, for example, (C₁-C₁₅)aliphatic, (C₁-C₁₀)aliphatic, (C₁-C₆)aliphatic, (C₁-C₅)aliphatic or (C₁-C₃)aliphatic. “Aliphatic” can be saturated or contain one or more units of unsaturation. Examples of aliphatic include alkyl, alkenyl and alkynyl. In some embodiments, aliphatic is alkyl, alkenyl or alkynyl. In some aspects, aliphatic is alkyl. In some embodiments, aliphatic is cyclic, for example, (C₃-C₁₂)cycloaliphatic, (C₃-C₈)cycloaliphatic or (C₃-C₆)cycloaliphatic. In some embodiments, aliphatic is cycloalkyl, for example, (C₃-C₁₂)cycloalkyl, (C₃-C₈)cycloalkyl or (C₃-C₆)cycloalkyl.

“Alkyl” refers to a saturated, aliphatic, branched or straight-chain, monovalent, hydrocarbon radical having the specified number of carbon atoms. Thus, “(C₁-C₁₀)alkyl” means a radical having from 1-10 carbon atoms in a linear or branched arrangement. In some embodiments, alkyl is (C₁-C₂₅)alkyl, for example, (C₁-C₁₅)alkyl, (C₁-C₁₀)alkyl, (C₁-C₆)alkyl, (C₁-C₅)alkyl or (C₁-C₃)alkyl. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like.

“Alkenyl” refers to an aliphatic, branched or straight-chain, monovalent, hydrocarbon radical having the specified number of carbon atoms and at least one carbon-carbon double bond. Thus, “(C₂-C₁₀)alkenyl” means a radical having from 2-10 carbon atoms in a linear or branched arrangement. In some embodiments, alkenyl is (C₂-C₂₅)alkenyl, for example, (C₂-C₁₅)alkenyl, (C₂-C₁₀)alkenyl, (C₂-C₆)alkenyl, (C₂-C₅)alkenyl or (C₂-C₃)alkenyl. Examples of alkenyl groups include ethenyl, 2-propenyl, 1-propenyl, 2-methyl-1-propenyl, 1-butenyl, 2-butenyl, 1-pentenyl, 2-pentenyl, 3-pentenyl, allyl, 1,3-butadienyl, 1,3-dipentenyl, 1,4-dipentenyl, 1-hexenyl, 1,3-hexenyl, 1,4-hexenyl, 1,3,5-trihexenyl, 2,4-dihexenyl and the like.

“Alkynyl” refers to an aliphatic, branched or straight-chain, monovalent, hydrocarbon radical having the specified number of carbon atoms and at least one carbon-carbon triple bond. Thus, “(C₂-C₁₀)alkynyl” means a radical having from 2-10 carbon atoms in a linear or branched arrangement. In some embodiments, alkynyl is (C₂-C₂₅)alkynyl, for example, (C₂-C₁₅)alkynyl, (C₂-C₁₀)alkynyl, (C₂-C₆)alkynyl, (C₂-C₅)alkynyl or (C₂-C₃)alkynyl. Examples of alkynyl groups include ethynyl, 1-propynyl, 2-propynyl, 1-butynyl, 2-butynyl, 2-methyl-1-butynyl, 1-pentynyl, 2-pentynyl, 3-pentynyl, 3-methyl-1-pentynyl, 2-methyl-1-pentynyl, 1-hexynyl, 2-hexynyl, 3-hexynyl and the like.

“Cycloalkyl” refers to a saturated, aliphatic, monovalent, monocyclic or polycyclic, hydrocarbon ring radical having the specified number of ring atoms. Thus, “(C₃-C₆)cycloalkyl” means a ring radical having from 3-6 ring carbons. Typically, cycloalkyl is monocyclic. In some embodiments, cycloalkyl is (C₃-C₁₅)cycloalkyl, for example, (C₃-C₁₂)cycloalkyl, (C₃-C₈)cycloalkyl or (C₃-C₆)cycloalkyl. Cycloalkyl includes, but is not limited to, cyclopropyl, cyclobutyl, cyclopentyl, cyclohexyl, and the like.

“Cyclyl” refers to an aromatic or non-aromatic, monocyclic or polycyclic, hydrocarbon ring radical having the specified number of ring atoms, wherein one or more carbon atoms (e.g., 0, 1, 2, 3, 4 or 5 carbon atoms) in the ring system may be replaced with a heteroatom (e.g., N, S and/or O). Thus, “(C₃-C₁₅)cyclyl” or a 5- to 15-membered cyclyl refers to a ring radical having from 3-15 ring atoms. Cyclyl includes cyclic aliphatic, cyclic heteroaliphatic, aryl and heteroaryl, as those terms are used herein. In some embodiments, cyclyl is cyclic aliphatic or cyclic heteroaliphatic (e.g., cycloalkyl or heterocyclyl). In some embodiments, cyclyl is aryl or heteroaryl (e.g., (C₆-C₁₅)aryl, (C₅-C₁₅)heteroaryl).

“Aryl” refers to a monocyclic or polycyclic (e.g., bicyclic, tricyclic), carbocyclic, aromatic ring system having the specified number of ring atoms, and includes aromatic ring(s) fused to non-aromatic rings, as long as one of the fused rings is an aromatic hydrocarbon. Thus, “(C₆-C₁₅)aryl” means an aromatic ring system having from 6-15 ring atoms. In some embodiments, aryl is (C₆-C₂₀)aryl, for example, (C₆-C₁₅)aryl, (C₆-C₁₂)aryl or (C₆-C₁₀)aryl. Examples of aryl include phenyl, naphthyl and fluorenyl.

“Heteroatom” refers to an atom that is not carbon or hydrogen. Examples of heteroatoms include, but are not limited to, nitrogen, oxygen, sulfur, boron, silicon, and the like. In some embodiments, heteroatom is selected from nitrogen, oxygen and sulfur.

“Heteroaliphatic,” as used herein, refers to a non-aromatic, branched, straight-chain or cyclic, hydrocarbon radical having the specified number of carbon atoms, wherein at least one carbon atom has been replaced with a heteroatom (e.g., N, S and/or O). Thus, “(C₁-C₁₀)heteroaliphatic” refers to a heteroaliphatic radical having from one to 10 atoms. In some embodiments, heteroaliphatic is (C₁-C₂₅)heteroaliphatic, for example, (C₁-C₁₅)heteroaliphatic, (C₁-C₁₀)heteroaliphatic, (C₁-C₆)heteroaliphatic, (C₁-C₅)heteroaliphatic or (C₁-C₃)heteroaliphatic. “Heteroaliphatic” can be saturated or contain one or more units of unsaturation. Examples of heteroaliphatic include heteroalkyl and heterocyclyl. In some embodiments, heteroaliphatic is heteroalkyl. In some embodiments, heteroaliphatic is cyclic, for example, (C₃-C₁₂)heterocycloaliphatic, (C₃-C₈)heterocycloaliphatic or (C₃-C₆)heterocycloaliphatic. In some embodiments, heteroaliphatic is heterocyclyl, for example, (C₃-C₁₂)heterocyclyl, (C₃-C₈)heterocyclyl or (C₃-C₆)heterocyclyl.

“Heteroalkyl” refers to a saturated, branched or straight-chain, monovalent, hydrocarbon radical having the specified number of chain atoms, wherein at least one carbon atom in the chain has been replaced with a heteroatom (e.g., N, S and/or O). Thus, “(C₁-C₁₀)heteroalkyl” means a radical having from 1-10 chain atoms in a linear or branched arrangement. In some embodiments, heteroalkyl is (C₁-C₂₅)heteroalkyl, for example, (C₁-C₁₅)heteroalkyl, (C₁-C₁₀)heteroalkyl, (C₁-C₆)heteroalkyl, (C₁-C₅)heteroalkyl or (C₁-C₃)heteroalkyl.

“Heterocyclyl” refers to a saturated, aliphatic, monocyclic or polycyclic (e.g., bicyclic, tricyclic), monovalent, hydrocarbon ring system having the specified number of ring atoms, wherein at least one carbon atom in the ring system has been replaced with a heteroatom (e.g., N, S and/or O). Thus, “(C₃-C₆)heterocyclyl” means a heterocyclic ring system having from 3-6 ring atoms. A heterocyclyl can be monocyclic, fused bicyclic, bridged bicyclic or polycyclic, but is typically monocyclic. A heterocyclyl can contain 1, 2, 3 or 4 (e.g., 1) heteroatoms (e.g., independently selected from N, S and O). When one heteroatom is S, it can be optionally mono- or di-oxygenated (i.e., —S(O)— or —S(O)₂). In some embodiments, heterocyclyl is (C₃-C₁₅)heterocyclyl, for example, (C₃-C₁₂)heterocyclyl, (C₃-C₈)heterocyclyl or (C₃-C₆)heterocyclyl. Examples of monocyclic heterocyclyls include, but are not limited to, aziridine, azetidine, pyrrolidine, piperidine, piperazine, azepane, tetrahydrofuran, tetrahydropyran, morpholine, thiomorpholine, dioxide, oxirane and the like.

“Heteroaryl” refers to an optionally substituted, monocyclic or polycyclic (e.g., bicyclic, tricyclic), aromatic, hydrocarbon ring system having the specified number of ring atoms, wherein at least one carbon atom in the ring system has been replaced with a heteroatom (e.g., N, S and/or O). “Heteroaryl” includes heteroaromatic ring(s) fused to aryl rings and/or non-aromatic rings, as long as one of the fused rings is a heteroaromatic ring. Thus, “(C₅-C₁₅)heteroaryl” means a heterocyclic aromatic ring system having from 5-15 ring atoms consisting of carbon and one or more heteroatoms (e.g., N, S and/or O). A heteroaryl can contain one or more, for example, 1, 2, 3, 4, 5 or 6; 1, 2, 3, 4, or 5; 1, 2 or 3; or 1 or 2, independently selected heteroatoms. In some embodiments, heteroaryl is (C₅-C₂₀)heteroaryl, for example, (C₅-C₁₅)heteroaryl, (C₅-C₁₂)heteroaryl, (C₅-C₁₀)heteroaryl or (C₅-C₆)heteroaryl. Monocyclic heteroaryls include, but are not limited to, furan, oxazole, thiophene, triazole, triazene, thiadiazole, oxadiazole, imidazole, isothiazole, isoxazole, pyridazine, pyridine, pyrazine, pyrimidine, pyrrole, pyrazole, tetrazole and thiazole. Bicyclic heteroaryls include, but are not limited to, phthalimide (e.g., N-phthalimide, such as N-hydroxyphthalimide), indolizine, indole, isoindole, indazole, azaindole, carbazole, azaindazole, pyrazole, pyrazolopyridine (e.g., pyrazolo[4,3-b]pyridine), imidazopyridazine, imidazopyridine (e.g., imidazo[1,2-b]pyridine), pyrrolopyridine (e.g., pyrrolo[2,3-b]pyridine), benzimidazole, benzofuran, benzothiazole, purine, quinoline, isoquinoline, cinnoline, phthalazine, quinazoline, quinoxaline, naphthyridine and pteridine. In some embodiments, heteroaryl contains at least one ring nitrogen, as, for example, in triazole, triazene, thiadiazole, oxadiazole, imidazole, isothiazole, isoxazole, pyridazine, pyridine, pyrazine, pyrimidine, pyrrole, pyrazole, tetrazole, thiazole, phthalimide (e.g., N-phthalimide, such as N-hydroxyphthalimide), indolizine, indole, isoindole, indazole, azaindole, carbazole, azaindazole, pyrazole, pyrazolopyridine (e.g., pyrazolo[4,3-b]pyridine), imidazopyridazine, imidazopyridine (e.g., imidazo[1,2-b]pyridine), pyrrolopyridine (e.g., pyrrolo[2,3-b]pyridine), benzimidazole, benzothiazole, quinoline, isoquinoline, cinnoline, phthalazine, quinazoline, quinoxaline, naphthyridine or pteridine.

“Amino” refers to —NH₂.

“Alkylamino” refers to —N(H)alkyl, wherein alkyl is as described herein.

“Dialkylamino” refers to —N(alkyl)₂, wherein alkyl is as described herein. Each alkyl in a “dialkylamino” can be independently chosen, such that each alkyl in a dialkylamino can be the same or the alkyls in a dialkylamino can be different from one another.

“Halogen” and “halo” are used interchangeably herein and each refers to fluorine, chlorine, bromine, or iodine. In some embodiments, halogen is selected from chlorine, bromine or iodine. In some embodiments, halogen is selected from bromine or iodine. In some embodiments, halogen is bromine.

“Haloalkyl” refers to alkyl, as that term is described herein, substituted with one or more independently selected halo. “Haloalkyl” includes mono, poly, and perhaloalkyl groups, wherein each halogen is independently selected from fluorine, chlorine, bromine and iodine (e.g., fluorine, chlorine and bromine). In one aspect, haloalkyl is perhaloalkyl (e.g., perfluoroalkyl). Examples of haloalkyl include, but are not limited to, trifluoromethyl and pentafluoroethyl.

It is understood that substituents (e.g., R², R³, R²⁰) on the compounds described herein can be selected by one of ordinary skill in the art to provide compounds that are chemically stable and that can be readily synthesized by techniques known in the art, as well as those methods described herein. Combinations of substituents are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

A designated group is unsubstituted, unless otherwise indicated, e.g., by the term “substituted” or “optionally substituted,” or by provision of a variable that denotes allowable substituents for a designated group. When the term “substituted” precedes a designated group, it means that one or more hydrogens of the designated group are replaced with a suitable substituent. An “optionally substituted” group or “substituted or unsubstituted” group can be substituted, as that term is described herein, or unsubstituted. Unless otherwise indicated, when an “optionally substituted group” or “substituted or unsubstituted group” is substituted, the group can have a suitable substituent at each substitutable position of the group and, when more than one position in any given structure is substituted, the substituent can be the same or different at every position (e.g., each substituent can be independently selected). In some embodiments, an optionally substituted group is substituted with 0-5 independently selected suitable substituents, e.g., 0-3, 0, 1, 2, 3, 4 or 5 independently selected suitable substituents. Alternatively, an “optionally substituted” group or “substituted or unsubstituted” group can be unsubstituted.

Suitable monovalent substituents on a substitutable carbon atom of an “optionally substituted” group are independently halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o}₂; SiR^{o}₃; —(C₁₋₄ straight or branched)alkylene)O—N(R^{o})₂; —(C₁₋₄ straight or branched) alkylene)C(O)O—N(R^{o}₂; or —O(CH₂CH₂O)_{1-25(e.g., 1-15, 1-10, 1-5)}R^{o}, wherein each R^{o} may be substituted as defined below and is independently hydrogen, C₁₋₆ aliphatic, —CH₂Ph, —O(CH₂)₀₋₁Ph, or a 5-6-membered saturated, partially unsaturated, or aromatic ring having 0-4 independently selected heteroatoms, or, notwithstanding the definition above, two independent occurrences of R^{o}, taken together with their intervening atom(s), form a 3-12-membered saturated, partially unsaturated, or aromatic mono- or bicyclic ring having 0-4 independently selected heteroatoms, which may be substituted as defined below.

Suitable monovalent substituents on R^{o} (or the ring formed by taking two independent occurrences of R^{o} together with their intervening atoms), are independently halogen, —(CH₂)₀₋₂R·, —(haloR·), —(CH₂)₀₋₂OH, —(CH₂)₀₋₂OR·, —(CH₂)₀₋₂CH(OR·)₂; —O(haloR·), —CN, —N₃, —(CH₂)₀₋₂C(O)R·, —(CH₂)₀₋₂C(O)OH, —(CH₂)₀₋₂C(O)OR·, —(CH₂)₀₋₂SR·, —(CH₂)₀₋₂SH, —(CH₂)₀₋₂NH₂, —(CH₂)₀₋₂NHR·, —(CH₂)₀₋₂NR·₂, —NO₂, —SiR·₃, —OSiR·₃, —C(O)SR·, -(C₁₋₄ straight or branched alkylene)C(O)OR·, or —SSR· wherein each R· is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aromatic ring having 0-4 independently selected heteroatoms. Such divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aromatic ring having 0-4 independently selected heteroatoms. Divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aromatic ring having 0-4 independently selected heteroatoms.

Suitable substituents on the aliphatic group of R* include halogen, —R·, -(haloR·), —OH, —OR·, —O(haloR·), —CN, —C(O)OH, —C(O)OR·, —NH₂, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aromatic ring having 0-4 independently selected heteroatoms.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aromatic ring having 0-4 independently selected heteroatoms, or, notwithstanding the definition above, two independent occurrences of R^{†}, taken together with their intervening atom(s) form an unsubstituted 3-12-membered saturated, partially unsaturated, or aromatic mono- or bicyclic ring having 0-4 independently selected heteroatoms.

Suitable substituents on the aliphatic group of R^{†} are independently halogen, —R·, —(haloR·), —OH, —OR·, —O(haloR·), —CN, —C(O)OH, —C(O)OR·, —NH₂, —NHR·, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aromatic ring having 0-4 independently selected heteroatoms.

In some embodiments, an optionally substituted group is optionally substituted with, e.g., 0-5, such as 0-3, 0, 1, 2, 3, 4, 5, substituents selected from oxo, halo, (C₁-C₁₀)haloalkyl, (C₁-C₁₀)alkyl, —O-(C₁-C₁₀)haloalkyl or —C(O)R¹³, wherein R¹³, for each occurrence, is independently —OH, —O-(C₁-C₁₀)alkyl or —O-halo(C₁-C₁₀)alkyl. In some embodiments, an optionally substituted group (e.g., an aryl or heteroaryl) is optionally substituted with, e.g., 0-5, such as 0-3, 0, 1, 2, 3, 4, 5, substituents selected from halo, (C₁-C₁₀)haloalkyl, (C₁-C₁₀)alkyl, —O-(C₁-C₁₀)alkyl, —O-(C₁-C₁₀)haloalkyl or —C(O)R¹³, wherein R¹³, for each occurrence, is independently —OH, —O-(C₁-C₁₀)alkyl or —O-halo(C₁-C₁₀)alkyl. In some embodiments, an optionally substituted group (e.g., an aliphatic) is optionally substituted with, e.g., 0-5, such as 0-3, 0, 1, 2, 3, 4, 5, substituents selected from oxo or halo or —C(O)R¹³, wherein R¹³, for each occurrence, is independently —OH, —O-(C₁-C₁₀)alkyl or —O-halo(C₁-C₁₀)alkyl.

Various of the functional groups described herein are conveniently protected in preparation for and/or during the transformations (e.g., functionalizations, derivatizations) described herein, and/or to maintain stability (e.g., for storage). Accordingly, suitable substituents also include protecting groups, such as those described in detail in Wuts, P. G. M. Protecting Groups in Organic Synthesis, 5^{th} Ed., New York, John Wiley & Sons, 2014, the entirety of which is incorporated herein by reference.

Examples of suitably protected hydroxyl groups include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenyl sulfonyl)ethyl, vinyl, allyl, and p-nitrobenzyl carbonate. Examples of silyl ethers include trimethylsilyl, triethylsilyl, t-butyldimethylsilyl, t-butyldiphenylsilyl, triisopropylsilyl ether, and other trialkylsilyl ethers. Examples of alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Examples of mono-protected aminos include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Di-protected aminos include aminos that are substituted with two substituents independently selected from those described above as mono-protected aminos, and further include cyclic imides, such as phthalimide, maleimide, succinimide, and the like. Di-protected aminos also include pyrroles and the like, 2,2,5,5-tetramethyl-[1,2,5]azadisilolidine and the like, and azide.

Protected aldehydes include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl esters, wherein each group is optionally substituted. Additional protected carboxylic acids include oxazolines and ortho esters.

Protected thiols include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester.

In a particular embodiment, suitable substituents are selected from —(CH₂)₀₋₂R·, -(haloR·), —(CH₂)₀₋₂OH, —(CH₂)₀₋₂OR·, —O(haloR·), —CN, —N₃, —(CH₂)₀₋₂SR·, —(CH₂)₀₋₂SH, —(CH₂)₀₋₂NH₂, —(CH₂)₀₋₂NHR·, —(CH₂)₀₋₂NR·₂, or —NO₂, wherein each R· is unsubstituted or where preceded by “halo” is substituted only with one or more halogens, and is independently selected from C₁₋₄ aliphatic (e.g., C₁ aliphatic). In another embodiment, suitable substituents are selected from a protecting group or —(CH₂)₀₋₂R·, -(haloR·), —(CH₂)₀₋₂OH, —(CH₂)₀₋₂OR·, —O(haloR·), —CN, —N₃, —(CH₂)₀₋₂SR·, —(CH₂)₀₋₂SH, —(CH₂)₀₋₂NH₂, —(CH₂)₀₋₂NHR·, —(CH₂)₀₋₂NR·₂ or —NO₂, wherein each R· is unsubstituted or where preceded by “halo” is substituted only with one or more halogens, and is independently selected from C₁₋₄ aliphatic (e.g., C₁ aliphatic). In yet another embodiment, suitable substituents are selected from a protecting group or halo, —OH or —NH₂. In yet another embodiment, suitable substituents are selected from halo, —OH or —NH₂.

The compounds described herein can be in the form of salts, for example, pharmaceutically acceptable salts. As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety.

Salts of the compounds described herein include salts derived from suitable inorganic and organic acids, and suitable inorganic and organic bases.

Examples of acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as various “solvates” or “hydrates.” A “hydrate” is a compound that exists in a composition with one or more water molecules. The composition can include water in stoichiometic quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Compounds described herein can also exist as various solids, such as crystalline solids.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. In all provided structures, any hydrogen atom can also be independently selected from deuterium (²H), tritium (³H) and/or fluorine (F). Such compounds are useful, for example, as analytical tools, as probes in biological assays, or as therapeutic agents in accordance with the present invention.

Compounds disclosed herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers, with all possible isomers and mixtures thereof, including optical isomers, being included in the present invention. For clarity, the disclosed compounds are depicted using three-dimensional structural formulas. The three-dimensional structural formulas depicted herein are not intended, however, to indicate stereochemistry, particularly with respect to the configuration of the stereocenter at the 2-position of a BCP compound. When a depicted compound has one or more chiral centers, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s).

“R” and “S” or “+” and “−” are used to indicate the absolute configuration of substituents around one or more chiral carbon atoms, e.g., the carbon atom at the 2-position of a BCP compound.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$e e = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D4 … )}{D 1 + ( D 2 + D 3 + D 4 … )} \vert \times 100 \% ,$$

where D1 and (D2+D3+D4 . . . ) represent the respective fractions of each diastereomer in a mixture, such that D1+(D2+D3+D4 . . . )=1. A diastereomer may be present in a de of at least or about 50%, about 60%, about 70%, about 80%, about 90%, about 95%, about 98%, about 99% or about 99.9%.

As used herein, “solvent” refers to a liquid that serves as a medium for a chemical reaction or other procedure in which compounds are being manipulated (e.g., purification). Typically, the solvent in the methods disclosed herein is an organic solvent or water, or a combination thereof. Examples of organic solvents include polar, protic solvents (e.g., an alcohol such as methanol, ethanol, butanol, such as tent-butanol), polar aprotic solvents (e.g., acetonitrile, dimethylformamide, tetrahydrofuran, ethyl acetate, acetone, methyl ethyl ketone) or nonpolar solvents (e.g., diethyl ether).

As used herein, “amination” refers to a chemical process, whereby a covalent carbon-nitrogen bond is formed. Amination may be achieved using nucleophilic or electrophilic sources of the nitrogen in the carbon-nitrogen bond, for example, as described herein. It will be understood that the source of the nitrogen may be heteroaromatic (e.g., a heteroaromatic amine) or heteroaliphatic (e.g., a heteroaliphatic amine) or ammonia or ammonium. In preferred embodiments, the source of the nitrogen is heteroaromatic. In some embodiments, the source of the nitrogen is a compound comprising a nucleophilic nitrogen (e.g., a heteroaromatic amine). In some embodiments, the source of the nitrogen is a compound comprising an electrophilic nitrogen. In some embodiments, amination is achieved via an SN1 or SN2 reaction. In some embodiments, amination is achieved in the presence of an organic catalyst or a transition metal catalyst.

As used herein, “arylation” refers to a chemical process in which an aryl, as that term is used herein, is covalently attached to another moiety (e.g., a BCP, such as the C-2 of a BCP) via a carbon in the aromatic ring system of the aryl.

As used herein, “heteroarylation” refers to a chemical process in which a heteroaryl, as that term is used herein, is covalently attached to another moiety (e.g., a BCP, such as the C-2 of a BCP) via an atom in the aromatic ring system of the heteroaryl. In some embodiments, the heteroaryl is covalently attached to the other moiety via a carbon atom in the aromatic ring system of the heteroaryl, as when the heteroarylation is a C-heteroarylation. In some embodiments, the heteroaryl is a heteroaromatic amine, and is covalently attached to the other moiety via a nitrogen atom in the aromatic ring system of the heteroaryl, as when the heteroarylation is a N-heteroarylation.

2-Substituted Bicyclo[1.1.1]pentanes (BCPs)

BCP compounds are valued in materials science for their rigidity, resistance to longitudinal deformation (e.g., high Young's modulus), lack of chemical reactivity, transparency (e.g., under ultraviolet and visible light) and thermal stability (e.g., up to 300° C.). Accordingly, BCPs are found in products as diverse as polymers, liquid crystals, monolayers and supramolecular structures, such as molecular rods and rotors. See, for example, Synthesis 2020, 52, 3295-3325; Chem. Eur. J. 2019, 25, 4590-4647; Angew. Chem. Int. Ed. 2017, 56, 5684-5718; and Chem. Rev. 2000, 100, 169-234, the entire contents of which are incorporated herein by reference.

In the pharmaceutical industry, BCP compounds can be used instead of small aromatic rings, such as mono- and para-substituted phenyls, and tent-butyl and alkynyl groups to increase the solubility, metabolic stability, permeability and partition coefficient of pharmaceuticals. It is believed that installation of a non-hydrogen group, such as fluorine, at the 2-position of BCP compounds replacing a phenyl group in a pharmaceutical would provide similar interactions (e.g., pi-pi, cation-pi, dipole-pi) to those between the phenyl group and its target, a protein, for example. See, for example, Asian J. Org. Chem. 2020, 9, 8-22; Synthesis 2020, 52, 3295-3325; Org. Biomol. Chem. 2019, 17, 2839-2849; and Synlett 2019, 30, 1-11, the entire contents of which are incorporated herein by reference.

A first embodiment is a compound of the following structural formula:

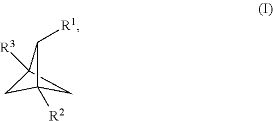

or a salt thereof, wherein:
R¹ is halo, —OH or —NH₂; and
R² and R³ are each independently —C(O)R¹⁰, —S(O)₂R¹⁰, —N(R¹¹)C(O)R¹⁰, —OC(O)R¹⁰, —H, halo, —OR¹¹, —N(R¹¹)R¹², aryl (and, in some aspects, (C₆-C₁₅)aryl), heteroaryl (and, in some aspects, (C₅-C₁₅)heteroaryl), —CH₂-halo, ⇒CH₂—OR¹¹, —CH₂—N(R¹¹)R¹², —C(H)(OR¹²)(NR¹¹R¹²) or —C(H)(OR¹¹)(OR¹²);
R¹⁰, for each occurrence, is independently —H, halo, —NR¹¹R¹², —OR¹¹, aliphatic (and, in some aspects, (C₁-C₁₀)aliphatic), heteroaliphatic (and, in some aspects, (C₁-C₁₀)heteroaliphatic), aryl (and, in some aspects, (C₆-C₁₅)aryl) or heteroaryl (and, in some aspects, (C₅-C₁₅)heteroaryl); and
R¹¹ and R¹², for each occurrence, are independently H, aliphatic (and, in some aspects, (C₁-C₁₀)aliphatic), heteroaliphatic (and, in some aspects, (C₁-C₁₀)heteroaliphatic), aryl (and, in some aspects, (C₆-C₁₅)aryl) or heteroaryl (and, in some aspects, (C₅-C₁₅)heteroaryl),
wherein each aliphatic, heteroaliphatic, aryl and heteroaryl is optionally and independently substituted.

In a first aspect of the first embodiment, R¹ is bromo, iodo, —OH or —NH₂. Values for the remaining variables are as described in the first embodiment.

In a second aspect of the first embodiment, R¹ is bromo or iodo. Values for the remaining variables are as described in the first embodiment, or first aspect thereof

In a third aspect of the first embodiment, R¹ is bromo. Values for the remaining variables are as described in the first embodiment, or first or second aspect thereof.

In a fourth aspect of the first embodiment:
R² and R³ are each independently —C(O)R¹⁰, —S(O)₂R¹⁰, —N(R¹¹)C(O)R¹⁰, —OC(O)R¹⁰, —H, halo, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², C(H)(OR¹²)(NR¹¹R¹²) or —C(H)(OR¹¹)(OR¹²);
R¹⁰, for each occurrence, is independently —H, halo, —NR¹¹R¹², —OR¹¹, (C₁-C₁₀)aliphatic, (C₁-C₁₀)heteroaliphatic, (C₆-C₁₅)aryl or (C₅-C₁₅)heteroaryl; and
R¹¹ and R¹², for each occurrence, are independently H, (C₁-C₁₀)aliphatic, (C₁-C₁₀)heteroaliphatic, (C₆-C₁₅)aryl or (C₅-C₁₅)heteroaryl,
wherein each aliphatic, heteroaliphatic, aryl and heteroaryl is optionally and independently substituted.
   Values for the remaining variables are as described in the first embodiment, or first through third aspects thereof.

In a fifth aspect of the first embodiment, R² is —C(O)R¹⁰, —S(O)₂R¹⁰, —N(R¹¹)C(O)R¹⁰, —OC(O)R¹⁰, —H, halo, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first embodiment, or first through fourth aspects thereof.

In a sixth aspect of the first embodiment, R³ is —C(O)R¹⁰, —S(O)₂R¹⁰, —N(R¹¹)C(O)R¹⁰, —OC(O)R¹⁰, halo, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first embodiment, or first through fifth aspects thereof.

In a seventh aspect of the first embodiment, R² and R³ are each independently —C(O)R¹⁰, —S(O)₂R¹⁰, —N(R¹¹)C(O)R¹⁰, —OC(O)R¹⁰, —H, halo, —OR¹¹, —N(R¹¹)R¹², (C₆-C₁₅)aryl or

(C₅-C₁₅)heteroaryl. Values for the remaining variables are as described in the first embodiment, or first through sixth aspects thereof.

In an eighth aspect of the first embodiment, R² and R³ are each independently —C(O)R¹⁰ or —H. Values for the remaining variables are as described in the first embodiment, or first through seventh aspects thereof.

In a ninth aspect of the first embodiment, R² and R³ are the same. Values for the variables, including R² and R³, are as described in the first embodiment, or first through eighth aspects thereof.

In a tenth aspect of the first embodiment, R² and R³ are different. Values for the variables, including R² and R³, are as described in the first embodiment, or first through eighth aspects thereof.

In an eleventh aspect of the first embodiment, R¹⁰, for each occurrence, is independently —H, halo, —NR¹¹R¹², —OR¹¹, (C₆-C₁₅)aryl or (C₅-C₁₅)heteroaryl. Values for the remaining variables are as described in the first embodiment, or first through tenth aspects thereof.

In a twelfth aspect of the first embodiment, R¹⁰, for each occurrence, is independently —OR¹¹. Values for the remaining variables are as described in the first embodiment, or first through eleventh aspects thereof.

In a thirteenth aspect of the first embodiment, R¹¹ and R¹², for each occurrence, are independently H, (C₆-C₁₅)aryl or (C₅-C₁₅)heteroaryl. Values for the remaining variables are as described in the first embodiment, or first through twelfth aspects thereof.

In a fourteenth aspect of the first embodiment, R¹¹, for each occurrence, is —H. Values for the remaining variables are as described in the first embodiment, or first through thirteenth aspects thereof.

In a fifteenth aspect of the first embodiment, R¹¹, for each occurrence, is —H or (C₁-C₁₀)alkyl. Values for the remaining variables are as described in the first embodiment, or first through fourteenth aspects thereof.

In a sixteenth aspect of the first embodiment, R¹ is halo. Values for the remaining variables are as described in the first embodiment, or first through fifteenth aspects thereof

In a seventeenth aspect of the first embodiment, R¹ is —OH. Values for the remaining variables are as described in the first embodiment, or first through sixteenth aspects thereof.

In an eighteenth aspect of the first embodiment, R¹ is —NH₂. Values for the remaining variables are as described in the first embodiment, or first through seventeenth aspects thereof.

In a nineteenth aspect of the first embodiment, the compound is not 2-chlorobicyclo[1.1.1]pentane, 1,2-dichlorobicylo[1.1.1]pentane, 2-chlorobicyclo[1.1.1]pentane-1,3-dicarboxylic acid, dimethyl 2-chlorobicyclo[1.1.1]pentane-1,3-dicarboxylate, 2-chloro-3-methoxycarbonylbicyclo[1.1.1]pentane-1-carboxylic acid, methyl 2-chloro-3-hydroxymethylbicyclo[1.1.1]pentane-1-carboxylate, methyl 2-chloro-3-formylbicyclo[1.1.1]pentane-1-carboxylate, 2-hydroxybicyclo[1.1.1]pentane, bicyclo[1.1.1]pentan-2-amine or dimethyl 2-fluorobicyclo[1.1.1]pentane-1,3-dicarboxylate, or a salt of any of the foregoing. Values for the variables are as described in the first embodiment, or any of the preceding aspects thereof.

A second embodiment is a compound represented by the following structural formula:

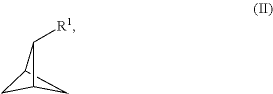

or a salt thereof. Values for the variables (e.g., R¹) are as described in the first embodiment, or any aspect thereof.

In a first aspect of the second embodiment, the compound is not 2-chlorobicyclo[1.1.1]pentane, 2-hydroxybicyclo[1.1.1]pentane or bicyclo[1.1.1]pentan-2-amine, or a salt of any of the foregoing. Values for the variables are as described in the first embodiment, or any aspect thereof.

A third embodiment is a compound represented by the following structural formula:

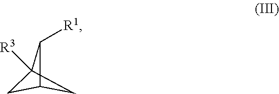

or a salt thereof. Values for the variables (e.g., R¹, R³) are as described in the first or fifth embodiment, or any aspect thereof.

In a first aspect of the third embodiment, R³ is —C(O)R¹⁰. Values for the remaining variables are as described in the first embodiment, or any aspect thereof.

In a second aspect of the third embodiment, the compound is not 1,2-dichlorobicylo[1.1.1]pentane, or a salt thereof. Values for the variables are as described in the first embodiment, or any aspect thereof, or the third embodiment, or any preceding aspect thereof.

A fourth embodiment is a compound represented by structural formula (I), or a salt thereof, wherein R² and R³ are each independently —C(O)R¹⁰, —S(O)₂R¹⁰, —N(R¹¹)C(O)R¹⁰, —OC(O)R¹⁰, halo, —OR¹¹, —N(R¹¹)R¹², aryl (and, in some aspects, (C₆-C₁₅)aryl), heteroaryl (and, in some aspects, (C₅-C₁₅)heteroaryl), —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first embodiment, or any aspect thereof.

In a first aspect of the fourth embodiment, R² is —C(O)R¹⁰, —S(O)₂R¹⁰, —N(R¹¹)C(O)R¹⁰, —OC(O)R¹⁰, halo, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first embodiment, or any aspect thereof.

In a second aspect of the fourth embodiment, R³ is —C(O)R¹⁰, —S(O)₂R¹⁰, —N(R¹¹)C(O)R¹⁰, —OC(O)R¹⁰, halo, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first embodiment, or any aspect thereof.

In a third aspect of the fourth embodiment, R² and R³ are each independently —C(O)R¹⁰. Values for the remaining variables are as described in the first embodiment, or any aspect thereof.

In a fourth aspect of the fourth embodiment, R² and R³ are the same. Values for the variables, including R² and R³, are as described in the first embodiment, or any aspect thereof, or the fourth embodiment, or first through third aspects thereof.

In a fifth aspect of the fourth embodiment, R² and R³ are different. Values for the variables, including R² and R³, are as described in the first embodiment, or any aspect thereof, or the fourth embodiment, or first through third aspects thereof.

In a sixth aspect of the fourth embodiment, the compound is not 2-chlorobicyclo[1.1.1]pentane-1,3-dicarboxylic acid, dimethyl 2-chlorobicyclo[1.1.1]pentane-1,3-dicarboxylate, 2-chloro-3-methoxycarbonylbicyclo[1.1.1]pentane-1-carboxylic acid, methyl 2-chloro-3-hydroxymethylbicyclo[1.1.1]pentane-1-carboxylate, methyl 2-chloro-3-formylbicyclo[1.1.1]pentane-1-carboxylate or dimethyl 2-fluorobicyclo[1.1.1]pentane-1,3-dicarboxylate, or a salt of any of the foregoing. Values for the variables are as described in the first embodiment, or any aspect thereof, or the fourth embodiment, or any preceding aspect thereof.

A fifth embodiment is a compound of structural formula (I), or a salt thereof, wherein:
R¹ is halo, —OH or —NH₂; and
R² and R³ are each independently —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², aryl (and, in some aspects, (C₆-C₁₅)aryl), heteroaryl (and, in some aspects, (C₅-C₁₅)heteroaryl), —CH₂-halo, —CH₂—OR¹¹, —CH₂—N(R¹¹)R¹², —C(H)(OR¹²)(NR¹¹R¹²) or —C(H)(OR¹¹)(OR¹²);
R¹⁰, for each occurrence, is independently —H, halo, —NR¹¹R¹², —OR¹¹, —O—NR¹¹R¹², aliphatic (and, in some aspects, (C₁-C₁₀)aliphatic), heteroaliphatic (and, in some aspects, (C₁-C₁₀)heteroaliphatic), aryl (and, in some aspects, (C₆-C₁₅)aryl) or heteroaryl (and, in some aspects, (C₅-C₁₅)heteroaryl); and
R¹¹ and R¹², for each occurrence, are independently H, aliphatic (and, in some aspects, (C₁-C₁₀)aliphatic), heteroaliphatic (and, in some aspects, (C₁-C₁₀)heteroaliphatic), aryl (and, in some aspects, (C₆-C₁₅)aryl) or heteroaryl (and, in some aspects, (C₅-C₁₅)heteroaryl), or taken together with the atoms to which they are attached and any intervening atoms, form a 3-15-membered cyclyl,
wherein each aliphatic, heteroaliphatic, aryl, heteroaryl and cyclyl is optionally and independently substituted.
   Alternative values for the variables are as described in the first through fourth embodiments, or any aspect or combination of aspects of the foregoing.

In a first aspect of the fifth embodiment, R m , for each occurrence, is independently —OR¹¹ or —O—NR¹¹R¹². Values for the remaining variables are as described in the first through fourth embodiments, or any aspect or combination of aspects of the foregoing, or the fifth embodiment.

In a second aspect of the fifth embodiment, —O—NR¹¹R¹² is —O—N-phthalimidyl or —O—N-(2-thiopyridonyl), each of which is optionally and independently substituted. Values for the remaining variables are as described in the first through fourth embodiments, or any aspect or combination of aspects of the foregoing, or the fifth embodiment, or first aspect thereof

In a third aspect of the fifth embodiment, R² is —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², C(H)(OR¹²)(NR¹¹R¹²) or —C(H)(OR¹¹)(OR¹²). Values for the remaining variables are as described in the first through fourth embodiments, or any aspect or combination of aspects of the foregoing, or the fifth embodiment, or first or second aspect thereof.

In a fourth aspect of the fifth embodiment, R³ is —C(O)R¹⁰, —S(O)₂R¹⁰, —N(R¹¹)C(O)R¹⁰, —OC(O)R¹⁰, halo, —CN, —OR¹¹, —N(R¹¹)_{R}¹², (C₆-C₁₅)aryl, (C₅-C₁₅)heteroaryl, —CH₂-halo, —CH₂—OR¹¹, —CH₂-N(R¹¹)R¹², —C(H)(OR¹²)(NR¹¹R¹²) or —C(H)(OR¹¹)(OR¹²). Values for the remaining variables are as described in the first through fourth embodiments, or any aspect or combination of aspects of the foregoing, or the fifth embodiment, or first through third aspects thereof.

In a fifth aspect of the fifth embodiment, R² is —H; and R³ is —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first through fourth embodiments, or any aspect or combination of aspects of the foregoing, or the fifth embodiment, or first through fourth aspects thereof.

In a sixth aspect of the fifth embodiment, R² and R³ are each independently —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl or (C₅-C₁₅)heteroaryl. Values for the remaining variables are as described in the first through fourth embodiments, or any aspect or combination of aspects of the foregoing, or the fifth embodiment, or first through fifth aspects thereof.

In a seventh aspect of the fifth embodiment, R² is —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first through fourth embodiments, or any aspect or combination of aspects of the foregoing, or the fifth embodiment, or first through sixth aspects thereof.

In an eighth aspect of the fifth embodiment, R² is —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²); and R³ is —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²). Values for the remaining variables are as described in the first through fourth embodiments, or any aspect or combination of aspects of the foregoing, or the fifth embodiment, or first through seventh aspects thereof.

In a ninth aspect of the fifth embodiment, —O—NR¹¹R¹² is a redox-active ester, such as —O—N-phthalimidyl or —O—N-(2-thiopyridonyl). Values for the remaining variables are as described in the first through fourth embodiments, or any aspect or combination of aspects of the foregoing, or the fifth embodiment, or first through eighth aspects thereof.

In a tenth aspect of the fifth embodiment, the compound is not 2-chlorobicyclo[1.1.1]pentane, 1,2-dichlorobicylo[1.1.1]pentane, 2-chlorobicyclo[1.1.1]pentane-1,3-dicarboxylic acid, dimethyl 2-chlorobicyclo[1.1.1]pentane-1,3-dicarboxylate, 2-chloro-3-methoxycarbonylbicyclo[1.1.1]pentane-1-carboxylic acid, methyl 2-chloro-3-hydroxymethylbicyclo[1.1.1]pentane-1-carboxylate, methyl 2-chloro-3-formylbicyclo[1.1.1]pentane-1-carboxylate, 2-hydroxybicyclo[1.1.1]pentane, bicyclo[1.1.1]pentan-2-amine or dimethyl 2-fluorobicyclo[1.1.1]pentane-1,3-dicarboxylate, or a salt of any of the foregoing. Values for the variables are as described in the in the first through fourth embodiments, or any aspect or combination of aspects of the foregoing, or the fifth embodiment, or any of the preceding aspects thereof.

The 2-substituted BCP compounds described herein can be synthesized and derivatized using methods set forth herein, as well as techniques known in the art. It will be understood that certain substituents on the BCP compounds described herein may undesirably interfere with one or more of the methods described herein. For example, and without wishing to be bound by any particular theory, subjecting a BCP compound having an exocyclic, sp³-hybridized, hydrogen-bearing carbon vicinal to a bridgehead carbon of the BCP could undesirably interfere with the desired functionalization of the bridging carbon due to the relative strength of the C—H bonds of the bridging carbon and the C—H bond(s) of the exocyclic, sp³-hybridized, hydrogen-bearing carbon, which are thought to be weaker and, therefore, more reactive under the conditions described herein for making a compound represented by structural formula I, or a salt thereof. For example, the existence of two identical substituents, such as two carboxylic acids, in a 2-substituted BCP described herein, could interfere with selective derivatization of just one of the carboxylic acids.

Accordingly, substituents and combinations of substituents in the methods described herein are preferably those that are not only chemically stable, but also chemically compatible with the conditions to which the compound is being subjected and/or the desired transformation (e.g., selective functionalization of the 2-position of a BCP; selective decarboxylation of the 1-carboxylate of a 2-substituted BCP). For example, in some aspects of a method of making a 2-substituted BCP described herein, it may be desirable to provide a compound of structural formula I′, or a salt thereof, wherein the substituents do not contain sp³ carbon-hydrogen bonds.

Protecting groups, such as those described herein, are often used to render otherwise chemically incompatible chemical moieties (e.g., substituent(s), functional group(s)) chemically compatible with a particular set of reaction conditions and/or a desired transformation. Accordingly, some aspects of any of the methods described herein further comprise protecting a chemically incompatible chemical moiety(ies) (e.g., substituent(s), functional group(s)) to form a protected chemical moiety(ies) (e.g., substituent(s), functional group(s)). Non-limiting examples of chemical moieties that can conveniently be protected and thereby rendered chemically compatible include hydroxyls, free aminos, aldehydes, thiols and carboxylic acids.

Deprotection of chemically incompatible chemical moiety(ies) (e.g., substituent(s), functional group(s)) results in removal of protecting group(s), and exposure of the original moity(ies). Accordingly, some aspects of any of the methods described herein further comprise deprotecting the protected chemical moiety(ies).

Orthogonal protecting group strategies can be employed when there are two or more chemical moieties in a compound that potentially share common reactivity and it is desired to derivatize or transform one (or more) chemical moiety(ies) independently of the one or more other chemical moiety(ies). Methods for protecting and deprotecting particular functional groups, as well as orthogonal protecting group strategies are known in the art and can be found, for example, in Wuts, P. G. M. Protecting Groups in Organic Synthesis, 5^{th} Ed., New York, John Wiley & Sons, 2014, the entirety of which is incorporated herein by reference.

### Methods of Making 2-Substituted BCPs

Also provided herein are methods of making 2-substituted BCPs, such as those described herein. In particular, provided herein are methods of functionalizing the 2-position of a BCP, for example, for further derivatization.

One embodiment is a method of making a compound of structural formula I, or a salt thereof, comprising reacting a compound of the following structural formula:

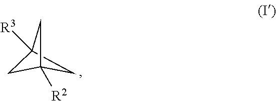

or a salt thereof, with a hydrogen atom abstractor and an R¹ source, thereby making the compound of structural formula I, or a salt thereof. Values for the variables (e.g., R², R³) are as described herein with respect to a compound of structural formula I, II or III, or a salt thereof, e.g., in the first through fifth embodiments, or any aspect of the foregoing.

For example, in an aspect of this embodiment, R² and R³ are each independently —C(O)R¹⁰, —S(O)₂R¹⁰, —N(R¹¹)C(O)R¹⁰, —OC(O)R¹⁰, —H, halo, —OR¹¹, —N(R¹¹)R¹², aryl, heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²); R¹⁰, for each occurrence, is independently —H, halo, —NR¹¹R¹², —OR¹¹, —O—NR¹¹R¹², aliphatic, heteroaliphatic, aryl or heteroaryl; and R¹¹ and R¹², for each occurrence, are independently H, aliphatic, heteroaliphatic, aryl or heteroaryl, or taken together with the atoms to which they are attached and any intervening atoms, form a 3-15-membered cyclyl, wherein each aliphatic, heteroaliphatic, aryl, heteroaryl and cyclyl is optionally and independently substituted. In a particular aspect of this aspect, R² and R³ are each independently —C(O)R¹⁰, —S(O)₂R¹⁰, —N(R¹¹)C(O)R¹⁰, —OC(O)R¹⁰, —H, halo, —OR¹¹, —N(R¹¹)R¹², aryl (and, in some aspects, (C₆-C₁₅)aryl) or heteroaryl (and, in some aspects, (C₅-C₁₅)heteroaryl); R¹⁰, for each occurrence, is independently —H, halo, —NR¹¹R¹², —OR¹¹, aryl (and, in some aspects, (C₆-C₁₅)aryl) or heteroaryl (and, in some aspects, (C₅-C₁₅)heteroaryl); and R¹¹ and R¹², for each occurrence, are independently H, aryl (and, in some aspects, (C₆-C₁₅)aryl) or heteroaryl (and, in some aspects, (C₅-C₁₅)heteroaryl), wherein each aryl and heteroaryl is optionally and independently substituted. In a particular aspect of this aspect, R² and/or R³ are not —H, for example, R² and R³ are each independently —C(O)R¹⁰, —S(O)₂R¹⁰, —N(R¹¹)C(O)R¹⁰, —OC(O)R¹⁰, halo, —OR¹¹, —N—(R¹¹)R¹², aryl (and, in some aspects, (C₆-C₁₅)aryl) or heteroaryl (and, in some aspects, (C₅-C₁₅)heteroaryl), wherein each aryl and heteroaryl is optionally and independently substituted. In another particular aspect of this aspect, R² is H and R³ is CO₂H, or R² and R³ are each CO₂H.

In another aspect of this embodiment, R² and R³ are each —H. In a more particular aspect of this aspect, the method comprises reacting bicyclo[1.1.1]pentane with the hydrogen atom abstractor and the R¹ source.

In yet another aspect of this embodiment, R² is —H and R³ is as described herein with respect to a compound of structural formula I, II or III, or a salt thereof, e.g., in the first through fifth embodiments, or any aspect of the foregoing. In a more particular aspect of this aspect, the method comprises reacting a compound of the following structural formula:

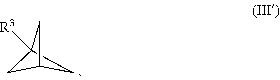

or a salt thereof, with the hydrogen atom abstractor and the R¹ source. Values for variable R³ in structural formula III′ are as described herein with respect to a compound of structural formula I, II or III, or a salt thereof, e.g., in the first through fifth embodiments, or any aspect of the foregoing. Preferred values for variable R³ in structural formula III′ are those wherein R³ is not —H.

As used herein, “hydrogen atom abstractor” refers to any reagent that is capable, under appropriate conditions, of abstracting a hydrogen free radical from a substrate. Hydrogen atom abstractors have been studied in the context of hydrogen atom transfer (HAT) reactions, where the hydrogen atom abstractor is typically either a photoreactive reagent (e.g., a photocatalyst) which, in its excited state, abstracts a hydrogen atom from a substrate directly, or a thermal reagent formed, for example, by interaction with a photocatalyst in its excited state. Without wishing to be bound by any particular theory, thermal hydrogen atom abstractors are thought to be formed by a mechanism that includes single electron transfer, energy transfer or proton-coupled electron transfer. Examples of hydrogen atom abstractors include, but are not limited to, N-chloro compounds, aromatic ketones (e.g., 4-benzoylpyridine, benzophenone, an anthraquinone, such as anthraquinone-2-sulfonate or anthraquinone-2-carboxylate, acetophenone), decatungstate anion and uranyl cation. Decatungstate anion and uranyl cation are typically supplied in salt form. Sources of decatungstate anion include tetrabutylammonium decatungstate, potassium decatungstate and sodium decatungstate.

In some aspects, the hydrogen atom abstractor is a catalyst, such as a photocatalyst.

In some aspects, the hydrogen atom abstractor is photoreactive, e.g., a N-chloro compound. In some aspects, the hydrogen atom abstractor is a thermal hydrogen atom abstractor.

In some aspects, the hydrogen atom abstractor is a N-chloro compound, such as N-chlorosuccinimide, 1,3-dichloro-5,5-dimethylimidazolidine-2,4-dione (also known as 1,3-dichloro-5,5-dimethylhydantoin), 2-chloroisoindoline-1,3-dione (also known as N-chlorophthalimide), 1,3,5-trichloro-1,3,5-triazinane-2,4,6-trione (also known as trichloroisocyanuric acid), N-chloro-N-(phenyl sulfonyl)benzenesulfonamide or N-chloro 4-methylbenzenesulfonamide. In some aspects, the hydrogen atom abstractor is an aromatic ketone, such as 4-benzoylpyridine, benzophenone, an anthraquinone, such as anthraquinone-2-sulfonate or anthraquinone-2-carboxylate, or acetophenone. In some aspects, the hydrogen atom abstractor is decatungstate anion. In some aspects, the hydrogen atom abstractor is uranyl cation.

An R¹ source should be capable, under appropriate conditions (e.g., conditions of a HAT reaction, conditions described herein and in the Exemplification), of reacting with a 2-BCP radical to form a carbon-R¹ bond at the 2-position of a BCP. It will be appreciated that the identity of the R¹ source will vary according to the value for R¹ to be introduced to the 2-position of the BCP substrate. For example, when the value for R¹ is halo, the R¹ source is conveniently a halogenating agent. Typically, an R¹ source is an electrophile.

In some aspects, the R¹ source is a halogenating agent, for example, a fluorinating agent, a chlorinating agent, a brominating agent or an iodinating agent. Non-limiting examples of chlorinating agents are N-chlorosuccinimide, 1,3-dichloro-5,5-dimethylimidazolidine-2,4-dione (also known as 1,3-dichloro-5,5-dimethylhydantoin), 2-chloroisoindoline-1,3-dione (also known as N-chlorophthalimide), 1,3,5-trichloro-1,3,5-triazinane-2,4,6-trione (also known as trichloroisocyanuric acid), N-chloro-N-(phenyl sulfonyl)benzenesulfonamide and N-chloro 4-methylbenzenesulfonamide. Non-limiting examples of brominating agents are bromotrichloromethane, tetrabromomethane, N-bromosuccinimide, N-bromophthalimide, 1,3-dibromo-5,5-dimethylimidazolidine-2,4-dione (known as 1,3-dibromo-5,5-dimethylhydantoin) and 2-bromobenzo[d]isothiazol-3(2H)-one 1,1-dioxide. Non-limiting examples of iodinating agents are perfluoroalkyliodide (e.g., nonafluoro-1-iodobutane), diiodomethane and N-iodosuccinimide.

It will be understood that, under certain conditions, a single reagent can be both hydrogen atom abstractor and R¹ source in the methods described herein. For example, N-chloro compounds often function as chlorinating agents. Such reagents are contemplated by this disclosure and the methods described herein. A person skilled in the art will be able to adjust the conditions of a reaction, such as by calibrating the number of equivalents of the dual-purpose reagent, to accommodate such scenarios.

In some aspects of this embodiment, the compound of structural formula I′ (e.g., BCP, a compound of structural formula III′), or a salt thereof, is reacted with the hydrogen atom abstractor and the R¹ source in a solvent, such as an organic solvent (e.g., acetonitrile), water or a mixture of an organic solvent (e.g., acetonitrile) and water.

In some aspects of this embodiment, reacting includes irradiating the hydrogen atom abstractor in the presence of the compound of structural formula I (e.g., BCP, a compound of structural formula III′), or a salt thereof, and the R¹ source. Both ultraviolet light (e.g., having a wavelength of from about 100 nm to about 400 nm) and blue light (e.g., having a wavelength of from about 400 nm to about 525 nm) have been found to mediate the HAT reactions described herein. Thus, in some aspects, irradiating comprises exposing to light having a wavelength of from about 100 nm to about 525 nm, for example, from about 365 nm to about 500 nm, or about 365 nm, about 390 nm, about 450 nm or about 500 nm. Sources of radiation include light- emitting diodes (LEDs), such as blue LEDs, and integrated photoreactors. Other sources of radiation, particularly of radiation having the wavelengths described above are known to those skilled in the art.

In some aspects of this embodiment, a method of making a 2-substituted BCP described herein (e.g., a compound of structural formula I, II or III, or a salt thereof) further comprises a method of derivatizing the 2-substituted BCP, for example, according to any of the methods of derivatizing 2-substituted BCPs described herein. For example, in some aspects wherein R² is H and R³ is CO₂H, or R² and R³ are each CO₂H, the method further comprises esterifying the carboxylic acid of R², R³ or R² and R³. Methods for esterifying carboxylic acids are known in the art and described hereinbelow.

It will be appreciated that certain methods of derivatization, such as the esterification depicted in Scheme 1, will result in a 2-substituted BCP described herein (e.g., a compound of structural formula I, II or III, or a salt thereof), while others will not. Both outcomes are contemplated by this disclosure.

### Methods of Derivatizing 2-Substituted BCPs

Also provided are methods of derivatizing 2-substituted BCPs, such as those described herein. In particular, provided herein are methods of derivatizing the 2-position of a BCP, for example, for elaboration into a pharmaceutical or a polymer, liquid crystal, monolayer or supramolecular structure, such as a molecular rod or rotor.

One embodiment is a method of derivatizing a 2-substituted BCP, such as a 2-substituted BCP described herein (e.g., a compound of structural formula I, II or III, or a salt thereof), comprising subjecting the 2-substituted BCP, or a suitably protected form thereof, to a derivatization reaction, thereby derivatizing the 2-substituted BCP. A derivatization reaction can be used to derivatize a 2-substituted BCP at one or more carbons, for example, at the 1-carbon, 2-carbon, 3-carbon, 1- and 2-carbons, 1- and 3-carbons or the 1-, 2- and 3-carbons.

A single derivatization reaction can be used to derivatize a single carbon of a 2-substituted BCP (e.g., the 1-carbon, 2-carbon, 3-carbon), particularly when the derivatization reaction is selective for the moiety (e.g., functional group, substituent, hydrogen) attached to the single carbon to be derivatized. A single derivatization reaction can alternatively be used to derivatize more than one carbon (e.g., the 1- and 2-carbons, 1- and 3-carbons or 1-, 2- and 3-carbons), particularly when the moieties (e.g., functional groups, substituents, hydrogen) attached to the more than one carbons to be derivatized are the same and/or share a common reactivity under the conditions of the particular derivatization reaction being used.

Multiple derivatization reactions (e.g., first and second derivatization reactions; first, second and third derivatization reactions) can be used to derivatize a single carbon of a 2-substituted BCP (e.g., the 1-carbon, 2-carbon, 3-carbon), as, for example, when a 1-carboxylic acid is esterified, then subjected to decarboxylative alkylation in a subsequent reaction. Multiple derivatization reactions (e.g., first and second derivatization reactions; first, second and third derivatization reactions) can also or alternatively be used to derivatize more than one carbon (e.g., the 1- and 2-carbons, 1- and 3-carbons or 1-, 2- and 3-carbons), as, for example, when the moieties (e.g., functional groups, substituents, hydrogens) attached to the more than one carbons to be derivatized lack common reactivity, for example, by virtue of the presence of a protecting group on one or more of the moieties or by virtue of inherently different (e.g., orthogonal) reactivities under the conditions of the particular derivatization reaction being used. A person skilled in the art will be able to order multiple derivatization reactions so as to obtain a desired derivative.

Herein, derivatization reactions are typically used to elaborate on the carbon skeleton of BCP without disrupting the connectivity of the carbon skeleton of BCP. Thus, as used herein, “derivatization reaction” refers to any reaction that converts one or more chemical moieties (e.g., functional group(s), substituent(s), hydrogen(s)) attached to the carbon skeleton of a BCP into a different chemical moiety.

It will be appreciated that a derivatization reaction should be selected to be compatible with the characteristics and identity of the compound to be derivatized as well as the desired transformation to be achieved. Thus, different derivatization reactions will be appropriate for different reactive chemical moieties and different desired derivatizations.

Preferred derivatization reactions involving carboxylates and/or carboxylic acids, e.g., at the 1- and/or 3-positions of a BCP, include esterification, decarboxylation and decarboxylative cross-coupling (e.g., decarboxylative arylation, decarboxylative heteroarylation, decarboxylative alkylation, decarboxylative amination, decarboxylative esterification).

Preferred derivatization reactions involving esters, e.g., at the 1- and/or 3-positions of a BCP, include decarboxylative cross-coupling (e.g., decarboxylative arylation, decarboxylative heteroarylation, decarboxylative alkylation, decarboxylative amination, decarboxylative esterification, decarboxylative etherification). Redox-active esters, such as N-hydroxy-phthalimide esters, thiohydroxamate esters (also known as Barton esters) and iodomesitylene dicarboxylates, are particularly suitable substrates in decarboxylative cross-couplings involving esters. Methods of making such esters, for example, from their corresponding carboxylic acids, are well-known in the art and described herein.

Thus, in some aspects wherein R² and/or R³ is —COOH, the method comprises esterifying the —COOH of R² and/or R³ to form an ester, for example, an alkyl (e.g., methyl, ethyl, propyl) ester or a redox-active ester (e.g., N-hydroxy-phthalimide ester, thiohydroxamate ester (also known as Barton esters) or iodomesitylene dicarboxylate). In further aspects, the method further comprises subjecting the ester to a decarboxylative cross-coupling. Methods of esterifying carboxylic acids are well-known in the art and described herein.

Derivatization reactions can be carried out using methods known in the art, as well as those described herein. For example, the following references describe methods for functionalizing and/or derivatizing a BCP, in particular, the 1- and/or 3-positions of a BCP: Ma. X. and Pham, L. N., Asian J. Org. Chem. 2020, 9, 8-22; Grover, N. and Senge, M. O., Synthesis 2020, 52, 3295-3325; and Kanazawa, J. and Uchiyama, M., Synlett 2019, 30, 1-11, the entire contents of which are incorporated herein by reference. A person skilled in the art will be able to appropriately order derivatization reaction(s) and/or protection and/or deprotection steps in order to obtain a desired derivative.

Preferred derivatization reactions involving chloro, bromo or iodo, e.g., at the 2-position of a BCP, include cross coupling (e.g., cross-electrophile coupling, such as cross-electrophile C—C coupling; spa C—N coupling). Such cross couplings have been used to derivatize the 2-position of a BCP described herein with an aryl or heteroaryl group (e.g., via arylation/heteroarylation), as well as a N-nucleophile (e.g., via amination), as described in the Exemplification.

For example, arylation and heteroarylation derivatization reactions involving cross coupling of chloro, bromo, or iodo, e.g., at the 2-position of a BCP, with an electrophilic aryl or heteroaryl source (such as an electrophilic R²⁰ source, as that variable is described herein) and N-heteroarylation reactions involving cross coupling of chloro, bromo, or iodo, e.g., at the 2-position of a BCP, with a heteroaromatic amine may be carried out in the presence of a halogen atom abstractor and a photocatalyst in a solvent, e.g., under photoredox conditions.

One embodiment is a method of making a compound of the following structural formula:

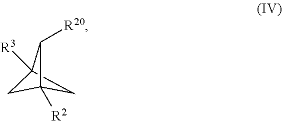

or a salt thereof, wherein R²⁰ is aryl (and, in some aspects, (C₆-C₁₅)aryl) or heteroaryl (and, in some aspects, (C₅-C₁₅)heteroaryl), each of which is optionally and independently substituted; and R² and R³ are as described herein with respect to a compound of structural formula I, II or III, or a salt thereof, e.g., in the first through fifth embodiments, or any aspect or combination of aspects of the foregoing. The method comprises reacting a compound of structural formula (I), or a salt thereof, wherein R¹ is halo, —OH or —NH₂, and R² and R³ are as described for the compound of structural formula (IV), with an R¹ activator, a photocatalyst and R²⁰-M in a solvent, wherein M is a transition metal (and, in some aspects, nickel or copper), thereby making a compound of structural formula (IV), or a salt thereof.

In a particular aspect of this embodiment, the (C₆-C₁₅)aryl or (C₅-C₁₅)heteroaryl of R²⁰ is optionally and independently substituted with one or more substituents selected from halo, halo(C₁-C₁₀)alkyl, —O-halo(C₁-C₁₀)alkyl or —C(O)R¹³, wherein R¹³, for each occurrence, is independently —OH, —O-(C₁-C₁₀)alkyl or —O-halo(C₁-C₁₀)alkyl. In some aspects, R² and R³ are each independently —C(O)R¹⁰ or —H; R¹⁰, for each occurrence, is independently —OR¹¹ or —O—NR¹¹R¹²; and R¹¹ and R¹², for each occurrence, are independently H or (C₁-C₁₀)alkyl, or taken together with the atoms to which they are attached and any intervening atoms, form a 3-15-membered cyclyl, which is optionally and independently substituted. In some aspects, —O—NR¹¹R¹² is —O—N-succinimidyl or —O—N-phthalimidyl, each of which is optionally and independently substituted.

An R¹ activator should be capable, under appropriate conditions (e.g., conditions of a derivatization reaction described herein and/or in the Exemplification), of activating the functional group at the 2-carbon of a BCP for derivatization. It will be appreciated that the identity of the le activator will vary according to the value for le to be activated. For example, when the value for R¹ is halo, the R¹ source is conveniently a halogen atom abstractor.

In some aspects, the R¹ activator is a halogen atom abstractor, and R¹ is halo. In some aspects wherein the activator is a halogen atom abstractor, R¹ is chloro, bromo or iodo, preferably, bromo.

As used herein, “halogen atom abstractor” refers to any reagent that is capable, under appropriate conditions (e.g., photoredox conditions), of abstracting a halogen free radical from a substrate. Halogen atom abstractors are typically photoreactive. Examples of halogen atom abstractors include, but are not limited to, silanes. Silanes containing a Si—H bond, such as tris(trimethylsilyl)silane, are thought to work by hydrogen atom abstraction of the Si—H bond, which forms the silyl radical. Other silanes useful in the context of the derivatization reactions described herein include silanes containing a Si—OH moiety, such as tris(trimethylsilyl)silanol, and silanes containing a Si—N bond, such as N-tris(trimethylsilyl)silyladamantan-1-amine, N-tris(trimethylsilyl)silyl-tert-butylamine and N-methyl-N-tris(trimethylsilyl)silyl-tent-butylamine. Without wishing to be bound by any particular theory, silanes, such as those described herein, are thought to be oxidized under photoredox conditions to form a silyl radical, which abstracts a halogen (e.g., a 2-bromide of a BCP described herein) to form a BCP radical (e.g., at the 2-carbon of the BCP). Reactivity of silanes containing a Si—H bond is described in J. Am. Chem. Soc. 2016, 138, 8084-8087, DOI: 10.1021/jacs.6b04818, the entire content of which is incorporated herein by reference in its entirety. Reactivity of silanes containing a Si—OH moiety is described in Science 360, 1010-1014 (2018), DOI: 10.1126/science.aat4133, the entire content of which is incorporated herein by reference in its entirety. Reactivity of silanes containing a Si—N bond is described in J. Am. Chem. Soc. 2020, 142, 11691-11697, DOI: 10.1021/jacs.0c04812, the entire content of which is incorporated herein by reference in its entirety. Tetrahedron 1997, 53, 8479-8490, the entire content of which is incorporated herein by reference in its entirety, describes the general concept of silyl radical nucleophilicity; and Chem. Rev. 2022, 122, 1485-1542 (in particular, section 2.1.5), the entire content of which is incorporated herein by reference in its entirety, provides a description of the mechanisms and expected reactivity of silyl radicals.

In some aspects, the halogen atom abstractor is a silane, e.g., tris(trimethylsilyl)silane, tris(trimethylsilyl)silanol or N-tris(trimethylsilyl)silyladamantan-1-amine.

Methods of making a compound of structural formula (IV), or a salt thereof, from a compound of structural formula (I), or a salt thereof, wherein R¹ is —OH or —NH₂ can be performed by replacing the halogen atom abstractor with an R¹ activator appropriate to these functional groups. For example, Nature 2021, 598, 451-456, the entire content of which is incorporated herein by reference in its entirety, discloses use of N-heterocyclic carbenes, or a salt thereof, to activate hydroxyl groups for metallaphotoredox-enabled deoxygenative arylation of alcohols. Thus, in some aspects of making a compound of structural formula (IV), or a salt thereof, from a compound of structural formula (I), or a salt thereof, wherein R¹ is —OH, the R¹ activator is a N-heterocyclic carbene, or a salt thereof. Examples of N-heterocyclic carbenes, or a salt thereof, suitable for use in accordance with the methods described herein include 5,7-di-tert-butyl-3-phenylbenzo[d]oxazol-3-ium tetrafluoroborate, 5,7-di-tert-butyl-3-(4-(trifluoromethyl)phenyl)benzo[d]oxazol-3-ium tetrafluoroborate and 5,7-di-tert-butyl-3-(4-(methoxy)phenyl)benzo[d]oxazol-3-ium tetrafluoroborate. These aspects are particularly useful for arylating or C-heteroarylating a 2—OH BCP.

J. Am. Chem. Soc. 2021, 143, 46, 19294-19299, the entire content of which is incorporated herein by reference, discloses use of electron-rich aldehydes to activate amines for dual nickel-photoredox-catalyzed deaminative cross-coupling of sterically hindered primary amines. Thus, in some aspects of making a compound of structural formula (IV), or a salt thereof, from a compound of structural formula (I), or a salt thereof, wherein R¹ is —NH₂, the R¹ activator is an electron-rich aldehyde. Examples of aldehydes suitable for use in accordance with the methods described herein include 2,4,6-trimethoxybenzaldehye. In some of these aspects, M in R²⁰-M is preferably nickel. These aspects are particularly useful for arylating or C-heteroarylating a 2-NH₂ BCP.

Org. Lett. 2019, 21, 9, 3346-3351, the entire content of which is incorporated herein by reference, discloses use of Katritzky salts to activate amines as pyridiniums, or a salt thereof, for deaminative reductive arylation enabled by nickel-photoredox dual catalysis. Thus, in some aspects of making a compound of structural formula (IV), or a salt thereof, from a compound of structural formula (I), or a salt thereof, wherein R¹ is —NH₂, the R¹ activator is a Katritzky salt. Examples of Katritzky salts suitable for use in accordance with the methods described herein include 2,4,6-triphenylpyrilium. In some of these aspects, M in R²⁰-M is preferably nickel. These aspects are particularly useful for arylating or C-heteroarylating a 2-NH₂BCP.

Examples of photocatalysts suitable for use in the derivatization reactions described herein include iridium-based photocatalysts, ruthenium-based photocatalysts and organic photocatalysts. Specific examples of photocatalysts include, but are not limited to, 1,2,3,5-tetrakis(carbazol-9-yl)-4,6-dicyanobenzene, 2,4,5,6-tetrakis(9H-carbazol-9-yl) isophthalonitrile, Ir(dF(CF₃))₂(dtbbpy)PF₆, Ir(ppy)₃, Ir(dFppy)₃, Ir(ppy)₂(dOMebpy)PF₆, Ir(ppy)₂(dtbbpy)PF₆, Ir(ppy)₂(bpy)PF₆, Ir(FMeppy)₂(dtbbpy)PF₆, Ir(dFMeppy)₂(dtbbpy)PF₆, Ir(dFMeppy)₂(bpy)PF₆, Ir(dF(F)ppy)₂(dtbbpy)PF₆, Ir(dF(CF₃)ppy)₂(bpy)PF₆, Ir(dF(CF₃)ppy)₂(4,4′-dFbpy)PF₆, Ir(dF(CF₃)ppy)₂(5,5′-dFbpy)PF₆ and Ru(bpy)₃C₁₂. Other photocatalysts are known in the art.

In some aspects, the photocatalyst is an iridium-based photocatalyst. In some aspects, the photocatalyst is an organic photocatalyst. In some aspects, the photocatalyst is 1,2,3,5-tetrakis(carbazol-9-yl)-4,6-dicyanobenzene, 2,4,5,6-tetrakis(9H-carbazol-9-yl) isophthalonitrile or Ir(dF(CF₃))₂(dtbbpy)PF₆).

In some aspects, reacting comprises irradiating the compound of structural formula (I), or a salt thereof, halogen atom abstractor (e.g., silane), photocatalyst and R²⁰-M in the solvent. In some aspects, irradiating comprises exposing to light having a wavelength of from about 100 nm to about 525 nm, for example, from about 365 nm to about 500 nm, or about 365 nm, about 390 nm, about 450 nm or about 500 nm. Sources of radiation include light-emitting diodes (LEDs), such as blue LEDs, and integrated photoreactors. Other sources of radiation, particularly of radiation having the wavelengths described above are known to those skilled in the art.

In some aspects, the method comprises reacting the compound of structural formula (I), or a salt thereof, with R²⁰-M in the further presence of a base. Examples of bases suitable for use in the derivatization reactions described herein include inorganic bases, such as Cs₂CO₃, Li(OtBu), Na₂CO₃, K₂CO₃, KOH, NaOH, K₃PO₄, Na₃PO₄, Na₂HPO₄, NaH₂PO₄, K₃PO₄, K₂HPO₄, KH₂PO₄, sodium acetate, potassium acetate, cesium acetate, sodium tert-butoxide and potassium tert-butoxide, and organic bases, in particular, organic amine bases, such as 1,5-diazabicyclo[4.3.0]non-5-ene (DBN), 2,6-lutidine, 1,8-diazabicylco(5.4.0)undec-7-ene (DBU), 1,1,3,3-tetramethylguanidine (TMG), N″-tert-butyl-N,N,N′,N′-tetramethylguanidine (BTMG), quinuclidine and 1,4-diazabicyclo[2.2.2]octane (DABCO). Superbases, such as tert-butylimino-tri(pyrrolidino)phosphorene (BTTP), are also suitable for use in the derivatization reactions described herein.

In some aspects, the method results in formation of a covalent carbon-carbon bond between 2-carbon of the compound of structural formula (I), or a salt thereof, and R²⁰, as when the method involves an arylation or C-heteroarylation. In further aspects of such aspects, the method further comprises contacting R²⁰—X, wherein X is halo (e.g., bromo) and is attached to a ring carbon of R²⁰, with a transition metal salt to form R²⁰-M.

In some aspects, the method results in formation of a covalent bond between 2-carbon of the compound of structural formula (I), or a salt thereof, and one ring nitrogen, as when the method involves N-heteroarylation. It will be understood that when the method describes an N-heteroarylation, R²⁰ is heteroaryl (e.g., (C₅-C₁₅)heteroaryl) containing at least one ring nitrogen. In further aspects of such aspects, the method further comprises contacting R²⁰—H with a transition metal salt to form R²⁰-M.

Examples of transition metal salts suitable for use in the derivatization reactions herein include nickel salts, such as Ni(dtbbpy)Br₂, Ni(dtbbpy)C₁₂, Ni(dOMebpy)Br₂ and Ni(dOMebpy)C₁₂, and copper salts, such as copper thiophene-2-carboxylate and copper bis(2,2,6,6-tetramethyl-3,5-heptanedionate).

In some aspects, the transition metal salt is a nickel salt. In some aspects, the transition metal salt is a copper salt.

R²⁰-M can also or alternatively be formed by contacting R²⁰—H (e.g., wherein H is attached to a ring nitrogen of R²⁰) or R²⁰—X (e.g., wherein X is attached to a ring carbon of R²⁰) with a transition metal complex which can in turn be formed, e.g., in situ, from a transition metal complex precursor and a ligand. Thus, for example, a nickel complex can be formed by contacting a nickel complex precursor, such as NiBr₂·glyme or NiCl₂·glyme, with a ligand, such as a 2,2′-bipyridine (bipy/bpy) ligand (e.g., 4,4′-di-tent-butyl-2,2′-bipyridine (dtbbpy), 4,4′-dimethoxy-2,2′-bipyridine (dOMebpy)). Likewise, a copper complex, typically, a copper(I) or copper(II) complex, can be formed by contacting a copper complex precursor, such as Cu(acetylacetonate)₂/Cu(MeCN₄)BF₄, CuCl₂ or CuCl, with a ligand, such as a bipy ligand (e.g., dOMebpy, bpy, 4,4′-dichloro-2,2′-bipyridine (dClbpy)) or acetoacetonate ligand.

In some aspects, the transition metal complex is a nickel complex. In some aspects, the transition metal complex is a copper complex.

From this discussion and the disclosure provided herein, it will be understood that in “R²⁰-M,” may, and likely does, have ligands that are not expressly defined by “R²⁰-M.” A person skilled in the art will be able to select appropriate ligands for the transformation being contemplated, at least in view of the disclosure provided herein.

The transition metal salts and complexes described herein can be used in stoichiometric or catalytic quantities in the derivatization reactions described herein, as described in the Exemplification.

In some aspects, there is a 1—C(O)O— and/or 3—C(O)O— group present in the BCP compound being derivatized (e.g., a compound of structural formula (I) and/or (IV)), or a salt thereof. For example, in some aspects, R² and/or R³ is —C(O)R¹⁰; R¹⁰ is —OR¹¹ or —O—NR¹¹R¹²; and R¹¹ and R¹², for each occurrence, are independently H or (C₁-C₁₀)alkyl, or taken together with the atoms to which they are attached and any intervening atoms, form a 3-15-membered cyclyl, e.g., —O—NR¹¹R¹² forms a redox-active ester, such as N-phthalimidyl. In some of such aspects, a method further comprises performing a decarboxylative cross-coupling before or after making the compound of structural formula (IV), or a salt thereof. For example, in some aspects, the method further comprises performing a decarboxylative cross-coupling on the compound of structural formula (I) and/or (IV), or a salt of either of the foregoing, or (I) and (IV), or a salt of the foregoing.

It will be appreciated that when a decarboxylative cross-coupling is performed on the 1- or 3-carbon of a compound of structural formula (I), or a salt thereof, the cross-coupling will result in a compound, or a salt thereof, wherein R² and/or R³ is different from the value of R² and/or R³, respectively, prior to the cross-coupling. Such a compound may still, nonetheless, be a compound of structural formula (I), or a salt thereof, as described herein or in the claims. Thus, R² and R³ in the compound prior to the cross-coupling may, but need not, have the same value as the compound resulting from the decarboxylative cross-coupling when the compound of structural formula (I), or a salt thereof, resulting from the decarboxylative cross-coupling is reacted with a halogen atom abstractor or other activator, photocatalyst and R²⁰-M in a solvent.

### EXEMPLIFICATION

2-Bromobicyclo[1.1.1]pentane-1,3-dicarboxylate derivatives can be synthesized according to Scheme 1, wherein R corresponds to R¹¹ as described in any of the embodiments, or aspects thereof, described herein.

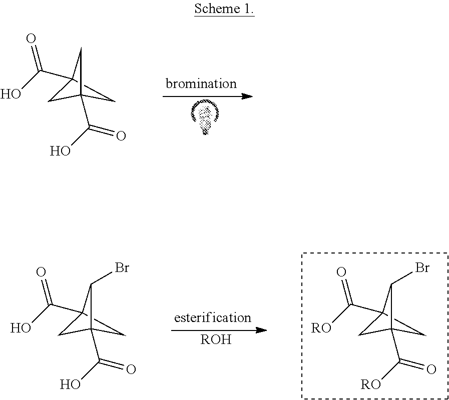

2-Bromobicyclo[1.1.1]pentane-1,3-dicarboxylic acid. To a 40 mL vial equipped with a stir bar was added commercially available bicyclo[1.1.1]pentane-1,3-dicarboxylic acid (4 mmol, 1 equiv.) and N-chlorosuccinimide (NCS, 12 mmol, 3 equiv.). Then, 20 mL of acetonitrile and 2 mL of deionized water was added, followed by the addition of bromotrichloromethane (BrCCl₃, 24 mmol, 6 equiv.). The solution was allowed to stir at room temperature for 1 minute (min) and then sonicated for 1 min until it became homogenous. Next, the solution was degassed by sparging with nitrogen for 5 min before capping and then sealing with parafilm. The reaction was stirred and irradiated using two 34 W blue LED lamps (3 cm away, with cooling fan on top) for 12 hours. The reaction mixture was removed from the light and cooled to ambient temperature. The reaction mixture was dried over Na₂SO₄, filtered, and then concentrated to evaporate all volatile components. The crude material was used in the next step directly. The reaction gave the product in 48% yield, based on crude ¹H NMR analysis with an internal standard.

Di-n-propyl 2-bromobicyclo[1.1.1]pentane-1,3-dicarboxylate. To a 250 mL round bottomed flask equipped with a stir bar was added 2-bromobicyclo[1.1.1]pentane-1,3-dicarboxylic acid (crude material, 16 mmol, 1 equiv) and 4-(dimethylamino)pyridine (DMAP, 32 mmol, 2 equiv.). Then, 1-propanol (n-PrOH, 640 mmol, 40 equiv.) and dichloromethane (DCM, 50 mL) was added, and the mixture was sonicated until the solution was homogeneous. N-(3-dimethylaminopropyl)-N′-ethylcarbodiimide hydrochloride (EDCI, 64 mmol, 4 equiv.) was then added portionwise, folllowed by another 50 mL of dichloromethane. The reaction was stirred for 16 hours under nitrogen at room temperature. Next, the solution was concentrated in vacuo until most of the DCM and n-PrOH was removed. Then, the mixture was diluted with 100 mL H₂O and 100 mL DCM, and the organic layer separated. The aqueous layer was extracted with DCM twice (100 mL each time). The combined organic layers were dried over Na₂SO₄, filtered and concentrated in vacuo. The crude material was purified using flash column chromatography (2:1 DCM:hexane) and the final product isolated as a colorless liquid (40% yield for two steps from bicyclo[1.1.1]pentane-1,3-dicarboxylic acid).

¹H NMR (500 MHz, CD₃CN) δ 4.63 (d, J=7.5 Hz, 1H), 4.05 (td, J=6.5, 1.0 Hz, 4H), 2.89 (dd, J=9.8, 2.9 Hz, 1H), 2.81 (d, J=2.7 Hz, 1H), 2.39 (dd, J=7.5, 3.0 Hz, 1H), 2.28 (dd, J=10.0, 2.7 Hz, 1H), 1.62 (h, J=7.3 Hz, 4H), 0.92 (t, J=7.4 Hz, 6H).

Dimethyl 2-bromobicyclo[1.1.1]pentane-1,3-dicarboxylate. To a 250 mL round bottomed flask equipped with a stir bar was added 2-bromobicyclo[1.1.1]pentane-1,3-dicarboxylic acid (crude material, 16 mmol, 1 equiv) and 4-(dimethylamino)pyridine (DMAP, 84 mmol, 6 equiv.). Then, methanol (MeOH, 1600 mmol, 100 equiv) and dichloromethane (DCM, 106 mL) was added, and the mixture was sonicated until the solution was homogeneous. N-(3-dimethylaminopropyl)-N′-ethylcarbodiimide hydrochloride (EDCI, 84 mmol, 6 equiv.) was then added portionwise, and the resultant solution sonicated until it was homogenous. The reaction was stirred for 16 hours under nitrogen at room temperature. Next, the solution was diluted with 100 mL H₂O and the organic layer separated. The aqueous layer was extracted with DCM twice (100 mL each time). The combined organic layers were dried over Na₂SO₄, filtered and concentrated in vacuo. The crude material was purified using flash chromatography (9:1 DCM:hexane) and the final product isolated as a pale yellow liquid (40% yield for two steps from bicyclo[1.1.1]pentane-1,3-dicarboxylic acid).

¹H NMR (500 MHz, CDCl₃) δ 4.56 (d, J=7.5 Hz, 1H), 3.74 (s, 6H), 3.04 (dd, J=10.1, 3.2 Hz, 1H), 2.83 (d, J=2.8 Hz, 1H), 2.44 (dd, J=7.5, 3.1 Hz, 1H), 2.32 (dd, J=9.2, 2.8 Hz, 1H).

2-Bromobicyclo[1.1.1]pentane-1-carboxylate derivatives can be synthesized according to Scheme 2, wherein R corresponds to R¹¹ as described in any of the embodiments, or aspects thereof, described herein.

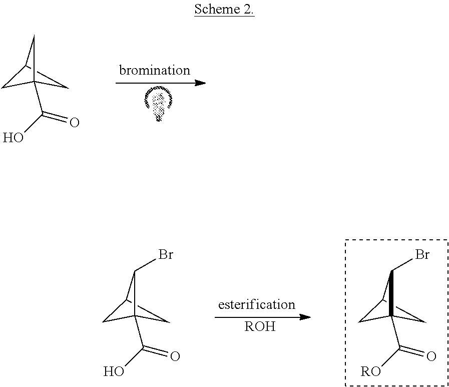

(±)-2-bromobicyclo[1.1.1]pentane-1-carboxylic acid. To a 8 mL vial equipped with a stir bar was added bicyclo[1.1.1]pentane-1-carboxylic acid (0.5 mmol, 1 equiv.) and N-chlorosuccinimide (NCS, 1 mmol, 2 equiv.). Then, 5 mL of acetonitrile was added, followed by the addition of bromotrichloromethane (BrCCl₃, 3 mmol, 6 equiv.). The solution was allowed to stir at room temperature for 1 min and then sonicated for 1 min until it became homogenous. Next, the solution was degassed by sparging with nitrogen for 3 min before capping and then sealing with parafilm. The reaction was stirred and irradiated using two 34 W blue LED lamps (3 cm away, with cooling fan on top) for 12 hours. The reaction mixture was removed from the light, cooled to ambient temperature. The reaction mixture was concentrated to evaporate all volatile component. The crude material was used in the next step directly.

(±)-Propyl 2-bromobicyclo[1.1.1]pentane-1-carboxylate. To a 40 mL vial equipped with a stir bar was added 2-bromobicyclo[1.1.1]pentane-1-carboxylic acid (crude material, 3 mmol, 1 equiv.), N-(3-dimethylaminopropyl)-N′-ethylcarbodiimide hydrochloride (EDCI, 6 mmol, 2 equiv.), 4-(dimethylamino)pyridine (DMAP, 3 mmol, 1 equiv.), and 1-propanol (30 mmol, 10 equiv.). Then, 20 mL of dichloromethane (DCM) was added. The solution was allowed to stir at room temperature for 5 min and then sonicated for 5 min until it became homogenous. The vial was capped and allowed to stir at room temperature for 12 h. Next, the reaction mixture was diluted with 20 mL H₂O and 20 mL DCM. The organic phase was collected and the aqueous phase was extracted with DCM twice (50 mL each time). The organic phase was combined and washed with brine (50 mL), dried over Na₂SO₄, filtered, and concentrated in vacuo. The crude material was purified using flash column chromatography (20:1 to 5:1 hexane:diethyl ether), and the final product was isolated as a colorless liquid (40% yield for two steps from bicyclo[1.1.1]pentane-1-carboxylic acid).

¹H NMR (500 MHz, CD₃CN) δ 4.49 (d, J=7.6 Hz, 1H), 4.02 (td, J=6.6, 1.4 Hz, 2H), 2.75 (dd, J=10.1, 2.9 Hz, 1H), 2.64 (s, 1H), 2.58 (d, J=2.8 Hz, 1H), 2.18 (dd, J=7.6, 2.9 Hz, 1H), 2.03 (dd, J=10.1, 2.8 Hz, 1H), 1.62 (h, J=7.3 Hz, 2H), 0.92 (t, J=7.4 Hz, 3H).

2-Bromobicyclo[1.1.1]pentane-1,3-dicarboxylate esters, such as dipropyl 2-bromobicyclo[1.1.1]pentane-1,3-dicarboxylate, can be derivatized according to Scheme 3.

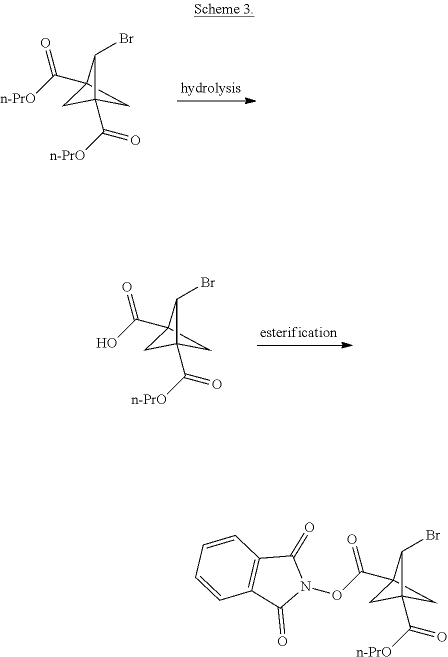

(±)-2-bromo-3-(propoxycarbonyl)bicyclo[1.1.1]pentane-1-carboxylic acid (P1) and 2-bromobicyclo[1.1.1]pentane-1,3-dicarboxylic acid (P2). To a 40 mL vial equipped with a stir bar was added dipropyl 2-bromobicyclo[1.1.1]pentane-1,3-dicarboxylate (3 mmol, 1 equiv.) and lithium hydroxide monohydrate (LiOH·H₂O, 3 mmol, 2 equiv.). Then, 15 mL dioxane and 15 mL deionized water were added. The solution was allowed to stir at room temperature for 90 min.

### Work-up Procedure for P1 and P2:

Step 1: The reaction mixture was diluted with 50 mL diethyl ether (Et₂O) and 50 mL H₂O. The Et₂O phase was then washed with Na₂CO₃ aqueous (aq.) solution (0.3 M, 10 mL), and all of the aqueous phases were combined. The Et₂O phase was dried over Na₂SO₄, filtered, and concentrated in vacuo to provide the starting material dipropyl 2-bromobicyclo[1.1.2]pentane-1,3-dicarboxylate in 20% yield.

Step 2: The combined aqueous phase from Step 1 was acidified with concentrated hydrochloric acid (HCl, 5 mL), and then extracted with DCM three times (40 mL each time). The combined DCM phase was then washed with deionized water three times (20 mL each time), and dried over Na₂SO₄, filtered, and concentrated in vacuo to provide P1 2-bromo-3-(propoxycarbonyl)bicyclo[1.1.1]pentane-1-carboxylic acid as a white solid in 60% yield.

P1 ¹H NMR (500 MHz, CD₃CN) δ 4.62 (d, J=7.5 Hz, 1H), 4.05 (t, J=6.4 Hz, 2H), 2.88 (dd, J=10.0, 3.0 Hz, 1H), 2.82 (d, J=2.7 Hz, 1H), 2.38 (dd, J=7.5, 3.0 Hz, 1H), 2.29 (dd, J=10.0, 2.7 Hz, 1H), 1.63 (h, J=7.3 Hz, 2H), 0.92 (t, J=7.4 Hz, 3H).

Step 3: The aqueous phases from Step 2 were combined and extracted with Et₂O three times (40 mL each time). The Et₂O phases were combined, dried over Na₂SO₄, filtered, and concentrated in vacuo to provide P2 2-bromobicyclo[1.1.1]pentane-1,3-dicarboxylic acid as a white solid in 20% yield.

P2 ¹H NMR (500 MHz, CD₃CN) δ 4.62 (d, J=7.5 Hz, 1H), 2.88 (dd, J=10.0, 3.0 Hz, 1H), 2.83 (d, J=2.8 Hz, 1H), 2.38 (dd, J=7.5, 3.0 Hz, 1H), 2.29 (dd, J=10.0, 2.8 Hz, 1H).

1-(1,3-dioxoisoindolin-2-yl) 3-propyl (1)-2-bromobicyclo[1.1.1]pentane-1,3-dicarboxylate. To a 40 mL vial equipped with a stir bar was added 2-bromo-3-(propoxycarbonyl)bicyclo[1.1.1]pentane-1-carboxylic acid (4 mmol, 1 equiv), N-hydroxyphthalimide (4.8 mmol, 1.2 equiv), and 4-(dimethylamino)pyridine (DMAP, 0.48 mmol, 0.2 equiv). Then, 20 mL DCM was added, followed by N,N′-diisopropylcarbodiimide (DIC, 4.8 mmol, 1.2 equiv). The vial was capped and allowed to stir at room temperature for 16 hours (h). Next, the reaction mixture was filtered to remove the precipitate, and the filtrate was concentrated in vacuo. The crude material was purified using flash column chromatography (4:1 to 3:1 hexane:ethyl acetate), and the final product was isolated as a white solid in 70% yield.

¹H NMR (500 MHz, CD₃CN) δ 7.93-7.86 (m, 4H), 4.86 (d, J=7 .5 Hz, 1H), 4.09 (t, J=6.4 Hz, 2H), 3.13 (dd, J=9.9, 3.1 Hz, 1H), 3.09 (d, J=2.8 Hz, 1H), 2.66 (dd, J=7.5, 3.1 Hz, 1H), 2.56 (dd, J=9.9, 2.8 Hz, 1H), 1.65 (h, J=7.4 Hz, 2H), 0.95 (t, J=7.4 Hz, 3H).

(±)-Propyl 2-bromobicyclo[1.1.1]pentane-1-carboxylate. To a 40 mL vial equipped with a stir bar was added 1-(1,3-dioxoisoindolin-2-yl)-3-propyl (±)-2-bromobicyclo[1.1.1]pentane-1,3-dicarboxylate (1 mmol, 1 equiv) and photocatalyst Ir(ppy)₂(dtbbpy)PF₆ (CAS 676525-77-2, commercially available from Sigma-Aldrich, Strem, TCI, and Oakwood, 0.01 mmol, 1 mol %). Then, 20 mL N,N-dimethylacetamide (DMA) was added, followed by triethylamine (Et₃N, 2 mmol, 2 equiv) and 1,4-cyclohexadiene (CHD, 2 mmol, 2 equiv). Next, the solution was degassed by sparging with nitrogen for 5 minutes before capping and then sealing the vial with parafilm. The reaction was stirred and irradiated using two 34 W blue LED lamps (3 cm away, with cooling fan on top) for 5 hours. The reaction mixture was then removed from the light, and allowed to cool to ambient temperature. Next, 40 mL of diethyl ether (Et₂O) was added, followed by 20 mL of deionized water. The organic phase was separated, and then washed with deionized water twice (20 mL each time) to remove most of the DMA. Next, the organic phase was dried over Na₂SO₄, filtered, and then concentrated to evaporate all volatile component. The crude material was purified using flash chromatography (10:1 hexane:diethyl ether), and the final product was isolated as a colorless liquid (approximately 70% yield).

¹H NMR (500 MHz, CD₃CN) δ 4.49 (d, J=7.6 Hz, 1H), 4.02 (td, J=6.6, 1.4 Hz, 2H), 2.75 (dd, J=10.1, 2.9 Hz, 1H), 2.64 (s, 1H), 2.58 (d, J=2.8 Hz, 1H), 2.18 (dd, J=7.6, 2.9 Hz, 1H), 2.03 (dd, J=10.1, 2.8 Hz, 1H), 1.62 (h, J=7.3 Hz, 2H), 0.92 (t, J=7.4 Hz, 3H).

¹H NMR (126 MHz, CDCl₃) δ 166.7, 66.3, 62.1, 49.8, 49.6, 47.1, 34.9, 21.8, 10.2.

HRMS (ESI) m/z calcd. for C₉H₁₄BrO₂ ([M+H]⁺) 233.0172, found 233.0002.

Amination of 2-bromobicyclo[1.1.1]pentane-1,3-dicarboxylate esters, such as dimethyl 2-bromobicyclo[1.1.1]pentane-1,3-dicarboxylate, with an amine, such as a N-containing heteroaryl nucleophile, can be conducted according to the following example procedure by substituting the desired amine for 6-chloro-1H-pyrrolo[2,3-b]pyridine in the following example procedure.

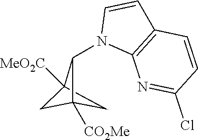 [SMILES: COC(=O)C12CC(C(=O)OC)(C1)C2n1ccc2ccc(Cl)nc21]

Dimethyl 2-(6-chloro-1H-pyrrolo[2,3-b]pyridin-1-yObicyclo[1.1.1]pentane-1,3-dicarboxylate. To five oven-dried 8 mL vials equipped with a Teflon stir bar were added 6-chloro-1H-pyrrolo[2,3-b]pyridine (7.5 mg, 0.05 mmol, 1.0 equiv.), copper bis(2,2,6,6-tetramethyl-3,5-heptanedionate) (Cu(TMHD)₂, 10.8 mg, 0.025 mmol, 0.5 equiv.), 1,5-diazabicyclo(4.3.0)non-5-ene (12.3 mg, 0.10 mmol, 2.0 equiv.), MeCN (1.5 mL, 0.03 M) and water (0.050 mL, 1.25 mmol, 25 equiv.). The resulting solutions were stirred for 1-2 minutes under air to ensure complete ligation of the nucleophile to the copper precatalyst. Following this complexation period, dimethyl 2-bromobicyclo[1.1.1]pentane-1,3-dicarboxylate (26.3 mg, 0.10 mmol, 2 equiv.) and tris(trimethylsilyl)silanol (33 mg, 0.13 mmol, 2.5 equiv.) were added to the mixture, after which the vial was capped and an 18 G vent needle was inserted through the Teflon-lined septum. The reaction mixture was subsequently stirred under air within the Integrated Photoreactor (450 nm irradiation) for 4 hours (25% light intensity, 2900 fan speed and 100 stir rate). After 4 hours, the reaction mixtures were combined into one 40 mL vial, diluted with ethyl acetate (EtOAc, 20 mL), followed by the addition of KF on alumina (40 wt. % from Sigma-Aldrich, 1.0 g) and tetrabutylammonium bromide (500 mg, 1.55 mmol, 6.2 equiv.) to the vial. This suspension was stirred under air for 2 hours, then filtered into a separatory funnel, using an additional 25 mL EtOAc wash to ensure complete transfer from the vial. The organic layer was subsequently washed with saturated Na2CO3 (10 mL), water (10 mL) and brine (10 mL), and the collected aqueous layer was extracted with EtOAc (10 mL). The combined organics were dried over NaSO₄ and concentrated to obtain the crude product. This residue was purified by flash column chromatography (0-20% EtOAc/hexane) on silica gel to afford the desired product (34.1 mg, 0.10 mmol, 41% yield).

¹H NMR (500 MHz, CDCl₃) δ 7.80 (d, J=8.2 Hz, 1H), 7.40 (d, J=3.6 Hz, 1H), 7.05 (d, J=8.2 Hz, 1H), 6.47 (d, J=3.6 Hz, 1H), 5.07 (d, J=6.5 Hz, 1H), 3.82 (s, 6H), 2.71 (dd, J=9.6, 3.5 Hz, 1H), 2.55-2.44 (m, 2H), 2.41 (dd, J=9.6, 2.6 Hz, 1H).

¹³C NMR (126 MHz, CDCl₃) δ 168.18, 147.54, 144.23, 130.92, 127.95, 118.78, 116.45, 100.91, 71.46, 52.33, 48.36, 46.82, 42.56.

HRMS (ESI) m/z calcd. for C₁₆H₁₅ClN₂O₇ ([M+1]⁺) 335.0793, found 335.0794.

Amination of 2-bromobicyclo[1.1.1]pentane-1,3-dicarboxylate esters, such as dimethyl 2-bromobicyclo[1.1.1]pentane-1,3-dicarboxylate, with a pyrazole or indazole can be conducted according to the following example procedure by substituting the desired pyrazole or indazole for 3-chloro-1H-indazole in the following example procedure.

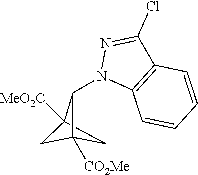 [SMILES: COC(=O)C12CC(C(=O)OC)(C1)C2n1nc(Cl)c2ccccc21]

Dimethyl 2-(3-chloro-1H-indazol-1-yl)bicyclo[1.1.1]pentane-1,3-dicarboxylate. To five oven-dried 8 mL vial equipped with a Teflon stir bar were added 3-chloro-1H-indazole (7.5 mg, 0.05 mmol, 1.0 equiv.), Cu(TMHD)₂ (14.4 mg, 0.03 mmol, 0.6 equiv.), lithium tert-butoxide (LiOt-Bu, 12 mg, 0.15 mmol, 3.0 equiv.), MeCN (1.5 mL, 0.03 M) and water (0.050 mL, 1.25 mmol, 25 equiv.). The resulting solutions were stirred for 1-2 minutes under air to ensure complete ligation of the nucleophile to the copper precatalyst. Following this complexation period, dimethyl 2-bromobicyclo[1.1.1]pentane-1,3-dicarboxylate (26.3 mg, 0.10 mmol, 2 equiv.) and tris(trimethylsilyl)silanol (33 mg, 0.13 mmol, 2.5 equiv.) were added to the mixture, after which the vial was capped and an 18 G vent needle was inserted through the Teflon-lined septum. The reaction mixture was subsequently stirred under air within the Integrated Photoreactor (450 nm irradiation) for 4 hours (25% light intensity, 2900 fan speed and 100 stir rate). After 4 hours, the reaction mixtures were combined into one 40 mL vial, diluted with EtOAc (20 mL), followed by the addition of KF on alumina (40 wt. % from Sigma-Aldrich, 1.0 g) and tetrabutylammonium bromide (500 mg, 1.55 mmol, 6.2 equiv.) to the vial. This suspension was stirred under air for 2 hours, then filtered into a separatory funnel, using an additional 25 mL EtOAc wash to ensure complete transfer from the vial. The organic layer was subsequently washed with saturated Na₂CO₃ (10 mL), water (10 mL) and brine (10 mL), and the collected aqueous layer was extracted with EtOAc (10 mL). The combined organics were dried over NaSO₄ and concentrated to obtain the crude product. This residue was purified by flash chromatography (0-15% EtOAc/hexane) on silica gel to afford the desired product (31.0 mg, 0.09 mmol, 37% yield).

¹H NMR (500 MHz, CDCl₃) δ 7.66 (d, J=8.5 Hz, 1H), 7.52 (d, J=8.5 Hz, 1H), 7.43 (t, J=8.4, 6.9, 1.1 Hz, 1H), 7.23 (t, 1H), 5.08 (d, J=6.3 Hz, 1H), 3.72 (s, 6H), 3.11 (dd, J=9.8, 3.3 Hz, 1H), 2.52 (dd, J=6.4, 3.4 Hz, 1H), 2.46 (d, J=2.4 Hz, 1H), 2.37 (dd, J=9.7, 2.4 Hz, 1H).

¹³C NMR (126 MHz, CDCl₃) δ 168.00, 141.45, 133.81, 127.78, 121.89, 121.85, 119.76, 109.94, 72.53, 52.24, 48.68, 47.05, 43.02.

HRMS (ESI) m/z calcd. for C₁₆H₁₅C₁N₂O₇ ([M+1]⁺) 335.0793, found 335.0809.

Amination of 2-bromobicyclo[1.1.1]pentane-1,3-dicarboxylate esters, such as dimethyl 2-bromobicyclo[1.1.1]pentane-1,3-dicarboxylate, or 2-bromobicyclo[1.1.1]pentane-1-carboxylate esters with various heteroaromatic amines, such as pyrazole, indazole, azaindole, indole, carbazole, or azaindazole (shown in Scheme 4), can be conducted according to Scheme 4, wherein X and Y are each independently CH or N.

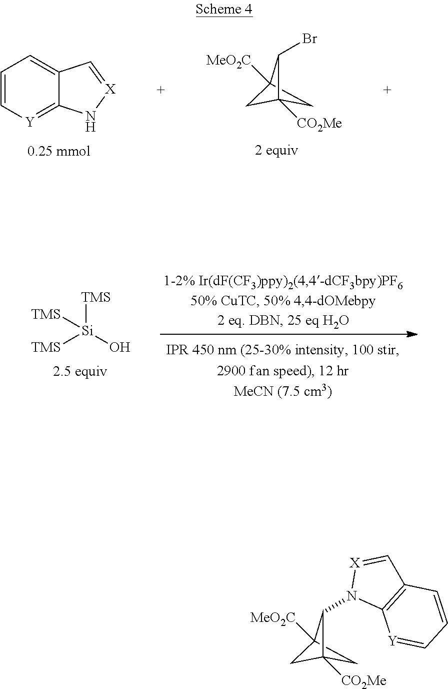

To an oven-dried, 40 mL vial equipped with a Teflon stir bar was added N-nucleophile (0.25 mmol, 1.0 equiv.), Copper(I)-thiophene-2-carboxylate (CuTC) (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]₂[4,4′-d(CF₃)bpy]PF₆ (0.002-0.004 mmol, 0.008-0.016 equiv.) and MeCN (7.5 mL, 0.03 M). 1,5-Diazabicyclo(4.3.0)non-5-ene (61.5 mg, 0.50 mmol, 2.0 equiv.) and water (0.13 mL, 6.25 mmol, 25 equiv.) were then added, and the resulting solution was stirred for 1-2 minutes under air to ensure complete ligation of the nucleophile to the copper precatalyst. Following this complexation period, brominated bicyclo[1.1.1]pentane (0.50 mmol, 2.0 equiv.) and supersilanol (0.17 mL, 0.63 mmol, 2.5 equiv.) were added to the mixture, after which the vial was capped and an 18 G vent needle was inserted through the Teflon-lined septum. The reaction mixture was subsequently stirred under air within the Integrated Photoreactor (450 nm irradiation) for 12 hours (25% light intensity, 2900 fan speed and 100 stir rate). After 12 hours, MeOH (0.25 mL) was added. This resulting solution was stirred under air for 2 hours, filtered through celite and concentrated in vacuo to obtain the crude product. The crude product was then purified by flash chromatography on silica gel to afford the desired product.

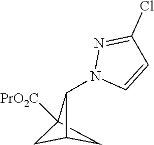

(±)-Propyl 2-(3-chloro-1H-pyrazol-1-yl)bicyclo[1.1.1]pentane-1-carboxylate. Prepared following Scheme 4 using 3-chloro-1H-pyrazole (25.6 mg, 0.25 mmol, 1.0 equiv.), CuTC (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]₂[4,4′-d(CF₃)bpy]PF₆ (2.3 mg, 0.002 mmol, 0.008 equiv.), 1,5-diazabicyclo(4.3.0)non-5-ene (61.5 mg, 0.50 mmol, 2.0 equiv.), propyl 2-bromobicyclo[1.1.1]pentane-1-carboxylate (116.6 mg, 0.50 mmol, 2.0 equiv.), supersilanol (0.17 mL, 0.63 mmol, 2.5 equiv.), water (0.13 mL, 1.25 mmol, 25 equiv.) and MeCN (7.5 mL, 0.03 M), and the Integrated Photoreactor (450 nm irradiation) for 12 hours (25% light intensity, 2900 fan speed and 100 stir rate). Purification by flash column chromatography (0-15% EtOAc/hexane) on silica gel afforded the desired product (52.6 mg, 83% yield) as a colorless oil.

¹H NMR (500 MHz, CDCl₃) δ 7.61 (d, J=2.4 Hz, 1H), 6.19 (d, J=2.4 Hz, 1H), 4.62 (d, J=6.4 Hz, 1H), 4.09 (t, J=6.8 Hz, 2H), 3.02 (s, 1H), 2.42 (dd, J=9.8, 3.3 Hz, 1H), 2.30 -2.14 (m, 2H), 1.99 (dd, J=9.8, 2.8 Hz, 1H), 1.68 (dt, J=7.6, 6.8 Hz, 2H), 0.94 (t, J=7.6 Hz, 3H).

¹C NMR (126 MHz, CDCl₃) δ 168.0, 139.5, 132.1, 105.3, 73.3, 66.9, 47.3, 47.2, 45.0, 32.8, 22.1, 10.6.

HRMS (ESI) m/z calcd. for C₁₂H₁₆C₁N₂O₂ ([M+1]⁺) 255.0895, found 255.0906.

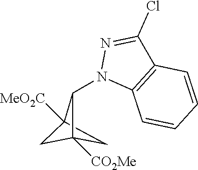 [SMILES: COC(=O)C12CC(C(=O)OC)(C1)C2n1nc(Cl)c2ccccc21]

Dimethyl 2-(3-chloro-1H-indazol-1-yl)bicyclo[1.1.1]pentane-1,3-dicarboxylate. Prepared following Scheme 4 using 3-chloro-1H-pyrazole (38.2 mg, 0.25 mmol, 1.0 equiv.), CuTC (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]₂[4,4′-d(CF₃)bpy]PF₆ (2.3 mg, 0.002 mmol, 0.008 equiv.), 1,5-diazabicyclo(4.3.0)non-5-ene (61.5 mg, 0.50 mmol, 2.0 equiv.), dimethyl 2-bromobicyclo[1.1.1]pentane-1,3-dicarboxylate (131.5 mg, 0.50 mmol, 2.0 equiv.), supersilanol (0.17 mL, 0.63 mmol, 2.5 equiv.), water (0.13 mL, 1.25 mmol, 25 equiv.) and MeCN (7.5 mL, 0.03 M), and the Integrated Photoreactor (450 nm irradiation) for 12 hours (25% light intensity, 2900 fan speed and 100 stir rate). Purification by flash column chromatography (0-15% EtOAc/hexane) on silica gel afforded the desired product (67.1 mg, 80% yield) as a colorless oil.

¹H NMR (500 MHz, CDCl₃) δ 7.66 (d, J=8.5 Hz, 1H), 7.52 (d, J=8.5 Hz, 1H), 7.43 (t, J=8.4, 6.9, 1.1 Hz, 1H), 7.23 (t, 1H), 5.08 (d, J=6.3 Hz, 1H), 3.72 (s, 6H), 3.11 (dd, J=9.8, 3.3 Hz, 1H), 2.52 (dd, J=6.4, 3.4 Hz, 1H), 2.46 (d, J=2.4 Hz, 1H), 2.37 (dd, J=9.7, 2.4 Hz, 1H).

¹³C NMR (126 MHz, CDCl₃) δ 168.00, 141.45, 133.81, 127.78, 121.89, 121.85, 119.76, 109.94, 72.53, 52.24, 48.68, 47.05, 43.02.

HRMS (ESI) m/z calcd. for C₁₆H₁₅C₁N₂O₇ ([M+1]⁺) 335.0793, found 335.0809.

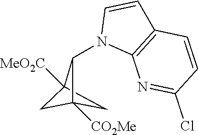 [SMILES: COC(=O)C12CC(C(=O)OC)(C1)C2n1ccc2ccc(Cl)nc21]

Dimethyl 2-(6-chloro-1H-pyrrolo[2,3-b]pyridin-1-yl)bicyclo[1.1.1]pentane-1,3-dicarboxylate. Prepared following Scheme 4 using 6-chloro-1H-pyrrolo[2,3-b]pyridine (38.2 mg, 0.25 mmol, 1.0 equiv.), CuTC (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]₂[4,4′-d(CF₃)bpy]PF₆ (2.3 mg, 0.002 mmol, 0.008 equiv.), 1,5-diazabicyclo(4.3.0)non-5-ene (61.5 mg, 0.50 mmol, 2.0 equiv.), dimethyl 2-bromobicyclo[1.1.1]pentane-1,3-dicarboxylate (131.5 mg, 0.50 mmol, 2.0 equiv.), supersilanol (0.17 mL, 0.63 mmol, 2.5 equiv.), water (0.13 mL, 1.25 mmol, 25 equiv.) and MeCN (7.5 mL, 0.03 M), and the Integrated Photoreactor (450 nm irradiation) for 12 hours (25% light intensity, 2900 fan speed and 100 stir rate). Purification by flash column chromatography (0-15% EtOAc/hexane) on silica gel afforded the desired product (50.0 mg, 60% yield) as a colorless oil.

¹H NMR (500 MHz, CDCl₃) δ 7.80 (d, J=8.2 Hz, 1H), 7.40 (d, J=3.6 Hz, 1H), 7.05 (d, J=8.2 Hz, 1H), 6.47 (d, J=3.6 Hz, 1H), 5.07 (d, J=6.5 Hz, 1H), 3.82 (s, 6H), 2.71 (dd, J=9.6, 3.5 Hz, 1H), 2.55-2.44 (m, 2H), 2.41 (dd, J=9.6, 2.6 Hz, 1H).

¹³C NMR (126 MHz, CDCl₃) δ 168.18, 147.54, 144.23, 130.92, 127.95, 118.78, 116.45, 100.91, 71.46, 52.33, 48.36, 46.82, 42.56.

HRMS (ESI) m/z calcd. for C₁₆H₁₅C₁N₂O₇ ([M+1]⁺) 335.0793, found 335.0794.

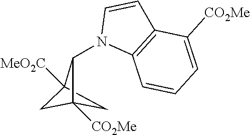 [SMILES: COC(=O)c1cccc2c1ccn2C1C2(C(=O)OC)CC1(C(=O)OC)C2]

Dimethyl 2-(4-(methoxycarbonyl)-1H-indo1-1-yl)bicyclo[1.1.1]pentane-1,3-dicarboxylate. Prepared following Scheme 4 using methyl 1H-indole-4-carboxylate (43.8 mg, 0.25 mmol, 1.0 equiv.), CuTC (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]₂[4,4′-d(CF₃)bpy]PF₆ (4.6 mg, 0.004 mmol, 0.016 equiv.), 1,5-diazabicyclo(4.3.0)non-5-ene (61.5 mg, 0.50 mmol, 2.0 equiv.), dimethyl 2-bromobicyclo[1.1.1]pentane-1,3-dicarboxylate (131.5 mg, 0.50 mmol, 2.0 equiv.), supersilanol (0.17 mL, 0.63 mmol, 2.5 equiv.), water (0.13 mL, 1.25 mmol, 25 equiv.) and MeCN (7.5 mL, 0.03 M), and the Integrated Photoreactor (450 nm irradiation) for 12 hours (25% light intensity, 2900 fan speed and 100 stir rate). Purification by flash column chromatography (0-15% EtOAc/hexane) on silica gel afforded the desired product (53.2 mg, 60% yield) as a white solid.

¹H NMR (500 MHz, CDCl₃) δ 7.91 (d, J=7.5 Hz, 1H), 7.67-7.53 (m, 1H), 7.40 (d, J=3.3 Hz, 1H), 7.24 (t, J=8.0 Hz, 1H), 7.14 (d, J=3.3 Hz, 1H), 5.12 (d, J=6.4 Hz, 1H), 3.98 (s, 3H), 3.69 (s, 6H), 3.01 (dd, J=9.6, 3.7 Hz, 1H), 2.51 (dd, J=6.4, 3.7 Hz, 1H), 2.46-2.34 (m, 2H).

¹³C NMR (126 MHz, CDCl₃) δ 168.1, 168.0, 137.7, 129.2, 128.4, 123.9, 121.9, 121.4, 114.9, 103.5, 73.2, 52.5, 52.0, 48.1, 47.1, 42.7.

HRMS (ESI) m/z calcd. for C₁₉H₂₀NO₆ ([M+1]⁺) 358.1285, found 358.1315.

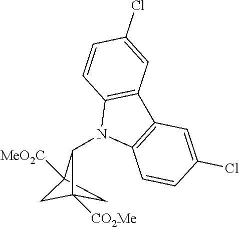 [SMILES: COC(=O)C12CC(C(=O)OC)(C1)C2n1c2ccc(Cl)cc2c2cc(Cl)ccc21]

Dimethyl 2-(3,6-dichloro-9H-carbazol-9-yl)bicyclo[1.1.1]pentane-1,3-dicarboxylate. Prepared following Scheme 4 using methyl 3,6-dichloro-9H-carbazole (59.0 mg, 0.25 mmol, 1.0 equiv.), CuTC (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]2[4,4′-d(CF₃)bpy]PF₆ (2.3 mg, 0.002 mmol, 0.008 equiv.), 1,5-diazabicyclo(4.3.0)non-5-ene (61.5 mg, 0.50 mmol, 2.0 equiv.), dimethyl 2-bromobicyclo[1.1.1]pentane-1,3-dicarboxylate (131.5 mg, 0.50 mmol, 2.0 equiv.), supersilanol (0.17 mL, 0.63 mmol, 2.5 equiv.), water (0.13 mL, 1.25 mmol, 25 equiv.) and MeCN (7.5 mL, 0.03 M), and the Integrated Photoreactor (450 nm irradiation) for 12 hours (25% light intensity, 2900 fan speed and 100 stir rate). Purification by flash column chromatography (0-15% EtOAc/hexane) on silica gel afforded the desired product (63.9 mg, 61% yield) as a white solid.

¹H NMR (500 MHz, CDCl₃) δ 7.96 (d, J=2.1 Hz, 2H), 7.37 (dd, J=8.8, 2.1 Hz, 2H), 7.30 (d, J=8.8 Hz, 2H), 5.13 (d, J=6.8 Hz, 1H), 3.66 (s, 6H), 3.19 (dd, J=9.7, 3.7 Hz, 1H), 2.68 (dd, J=6.8, 3.7 Hz, 1H), 2.56-2.38 (m, 2H).

¹³C NMR (126 MHz, CDCl₃) δ 168.4, 139.8, 127.0, 126.0, 124.1, 120.2, 111.2, 74.1, 52.6, 49.3, 47.9, 44.1.

HRMS (ESI) m/z calcd. for C₂₁H₁₈C₁₂NO₄ ([M+1]⁺) 418.0607, found 418.0614.

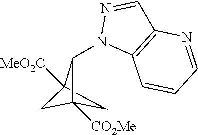 [SMILES: COC(=O)C12CC(C(=O)OC)(C1)C2n1ncc2ncccc21]

Dimethyl 2-(1H-pyrazolo[4,3-b]pyridin-1-yl)bicyclo[1.1.1]pentane-1,3-dicarboxylate. Prepared following Scheme 4 using 1H-pyrazolo[4,3-b]pyridine (29.8 mg, 0.25 mmol, 1.0 equiv.), CuTC (23.8 mg, 0.13 mmol, 0.5 equiv.), 4,4′-dimethoxy-2,2′-bipyridine (27.0 mg, 0.13 mmol, 0.5 equiv.), Ir[dF(CF₃)ppy]₂[4,4′-d(CF₃)bpy]PF₆ (4.6 mg, 0.004 mmol, 0.016 equiv.), 1,5-diazabicyclo(4.3.0)non-5-ene (61.5 mg, 0.50 mmol, 2.0 equiv.), dimethyl 2-bromobicyclo[1.1.1]pentane-1,3-dicarboxylate (131.5 mg, 0.50 mmol, 2.0 equiv.), supersilanol (0.17 mL, 0.63 mmol, 2.5 equiv.), water (0.13 mL, 1.25 mmol, 25 equiv.) and MeCN (7.5 mL, 0.03 M), and the Integrated Photoreactor (450 nm irradiation) for 12 hours (25% light intensity, 2900 fan speed and 100 stir rate). Purification by flash column chromatography (0-60% EtOAc/hexane) on silica gel afforded the desired product (55.0 mg, 73% yield) as a colorless oil.

¹H NMR (500 MHz, CDCl₃) δ 8.57 (dd, J=4.4, 1.2 Hz, 1H), 8.21 (d, J=1.0 Hz, 1H), 7.97 (dt, J=8.6, 1.2 Hz, 1H), 7.28 (dd, J=8.6, 4.4 Hz, 1H), 5.13 (d, J=6.4 Hz, 1H), 3.72 (s, 6H), 3.00 (dd, J=9.8, 3.3 Hz, 1H), 2.60-2.43 (m, 2H), 2.38 (dd, J=9.8, 2.4 Hz, 1H).

¹³C NMR (126 MHz, CDCl₃) δ 168.1, 146.1, 142.5, 134.7, 133.2, 120.9, 117.9, 72.4, 52.4, 48.9, 47.3, 43.0.

HRMS (ESI) m/z calcd. for C₁₅H₁₆N₃O₄ ([M+1]⁺) 302.1135, found 302.1145.

Arylation of 2-bromobicyclo[1.1.1]pentane-1,3-dicarboxylate esters, such as dimethyl 2-bromobicyclo[1.1.1]pentane-1,3-dicarboxylate, can be conducted according to the following example procedure by substituting the desired aryl for methyl 4-bromobenzoate in the following example procedure.

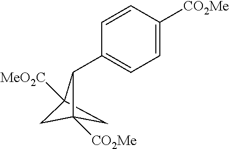 [SMILES: COC(=O)c1ccc(C2C3(C(=O)OC)CC2(C(=O)OC)C3)cc1]

Dimethyl 2-(4-(methoxycarbonyl)phenyl)bicyclo[1.1.1]pentane-1,3-dicarboxylate. To an 8 mL vial equipped with a stir bar was added methyl 4-bromobenzoate (10.8 mg, 0.05 mmol, 1 equiv.), dimethyl 2-bromobicyclo[1.1.1]pentane-1,3-dicarboxylate (26.3 mg, 0.1 mmol, 2 equiv.), N-tris(trimethylsilyl)silyladamantan-1-amine (39.8 mg, 0.1 mmol, 2 equiv.), and 2,6-lutidine (10.7 mg, 0.1 mmol, 2 equiv.). To a separate vial was added 1,2,3,5-tetrakis(carbazol-9-yl)-4,6-dicyanobenzene (4-CzIPN; 3.94 mg, 5 μmol, 0.1 equiv.) and 5 ml of acetonitrile as solvent. To another separate vial was added nickel(II) bis(2,2,6,6-tetramethyl-3,5-heptanedionate) (Ni(TMHD)₂, 10.63 mg, 0.025 mmol, 0.5 equiv.), 4,4′-di-methoxy-2,2′-bipyridine (5.95 mg, 0.0275 mmol, 0.55 equiv.), and 2.5 ml of acetonitrile as solvent. Both photocatalyst and nickel precatalyst stock solutions were sonicated for 5 minutes, after which time 0.5 mL of the photocatalyst solution (1 mol%, 0.5 μmol, 0.01 equiv.) and precatalyst solution (10 mol%, 5 μmol, 0.1 equiv) were syringed into the reaction vessel, respectively. The solution was degassed by sparging with nitrogen for 10 minutes before sealing with Parafilm. The reaction was stirred and irradiated with a 34 W blue LED lamp (6 cm away, with cooling fan to keep the reaction temperature at 25° C.) for 12 hours. The reaction was diluted with 50 mL dichloromethane and 50 mL Na₂CO₃, and the organic layer separated. The aqueous layer was extracted with dichloromethane three more times (3×25 mL). The combined organics were dried over MgSO₄, filtered through Celite and concentrated to obtain the crude product. This residue was purified by flash chromatography (15-20% EtOAc/hexane) on silica gel followed by preparative TLC (100% DCM) to afford the desired product (5.6 mg, 0.02 mmol, 35% yield).

¹H NMR (500 MHz, CDCl₃) δ 7.98 (d, J=8.0 Hz, 1H), 7.35 (d, J=8.0 Hz, 1H), 4.04 (d, J=6.7 Hz, 1H), 3.91 (s, 3H), 3.74 (s, 6H), 2.67 (dd, J=9.3, 3.1 Hz, 1H), 2.46-2.26 (m, 3H).

HRMS (ESI) m/z calcd. for C₁₇H₁₈O₆ ([M+1]⁺) 319.1176, found 319.1173.

Arylation/heteroarylation of 2-bromobicyclo[1.1.1]pentane-1,3-dicarboxylate esters, such as dimethyl 2-bromobicyclo[1.1.1]pentane-1,3-dicarboxylate, or 2-bromobicyclo[1.1.1]pentane-1-carboxylate esters can be conducted using (hetero)aryl halides, such as phenyl bromide or pyridylbromide (shown in Scheme 5), according to Scheme 5, wherein X is CH or N.

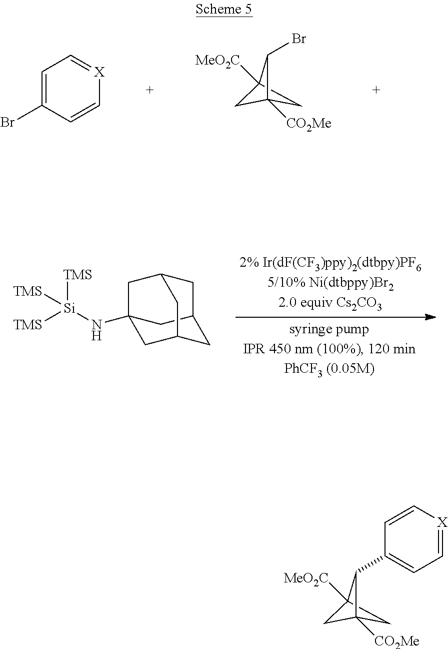

A 40 mL vial equipped with a cross-shaped stir bar was charged with dimethyl 2-bromobicyclo[1.1.1]pentane-1,3-dicarboxylate (263 mg, 1.00 mmol, 2.0 equiv), cesium carbonate (326 mg, 1.00 mmol, 2.0 equiv), Ir(dF(CF₃))₂(dtbbpy)PF₆ (11.2 mg, 10.0 μmol, 0.02 equiv) and Ni(dtbbpy)Br₂ (12.2 mg, 25.0 μmol, 0.05 equiv). The vial was closed, sealing the cap with parafilm, evacuated and filled with nitrogen three times followed by addition of trifluorotoluene (previously degassed by sparging with nitrogen for 15 minutes, 10 mL). The mixture was stirred at room temperature for 5 minutes, after which time the Ni(dtbbpy)Br₂ was fully dissolved. The vial was then placed in a water bath with a stir bar positioned above the Integrated Photoreactor and stirred under nitrogen for 2 hours (450 nm, 100% light intensity, 800 rpm stir rate, 5200 rpm fan rate). While stirring and irradiating the reaction mixture, a solution of (hetero)aryl halide (0.50 mmol) and N-tris(trimethylsilyl)silyladamantan-1-amine (318 mg, 0.80 mmol, 1.6 equiv) in trifluorotoluene (previously degassed by sparging with nitrogen for 15 minutes, 5 mL) was added via syringe pump (3 mL/hour, completion of addition after 100 minutes, followed by irradiation for 20 minutes without any further addition of (hetero)aryl halide solution). After 2 hours, the reaction mixture was removed from the water bath on top of the Integrated Photoreactor, opened to air and chloroform (approximately 10 mL) and methanol (300 μL) were added. The reaction mixture was stirred at room temperature for 60 minutes and then filtered through celite, rinsing with chloroform. The filtrate was concentrated in vacuo and the resulting crude material was purified by flash column chromatography to give the 2-arylated BCP compound.

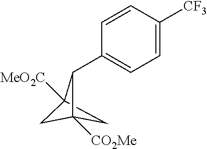 [SMILES: COC(=O)C12CC(C(=O)OC)(C1)C2c1ccc(C(F)(F)F)cc1]

Dimethyl 2-(4-(trifluoromethyl)phenyl)bicyclo[1.1.1]pentane-1,3-dicarboxylate. Prepared according to Scheme 5 by slowly adding a solution of 1-bromo-4-(trifluoromethyl)benzene (113 mg, 0.50 mmol) and silane in PhCF₃ via syringe pump to a solution of dimethyl 2-bromobicyclo[1.1.1]pentane-1,3-dicarboxylate (263 mg, 1.00 mmol, 2.0 equiv), cesium carbonate, Ir(dF(CF₃))₂(dtbbpy)PF₆ and 5% Ni(dtbbpy)Br₂ in PhCF₃. Purification via flash column chromatography (5-20% EtOAc/hexane) gave 139.3 mg (424 mmol, 85%) as a white solid.

¹H NMR (500 MHz, CDCl₃) δ 7.57 (d, J=8.1 Hz, 2H), 7.41 (d, J=8.2 Hz, 2H), 4.03 (d, J=6.7 Hz, 1H), 3.74 (s, 6H), 2.67 (dd, J=10.0, 3.1 Hz, 1H), 2.45-2.34 (m, 3H).

¹³C NMR (126 MHz, CDCl₃) δ 169.2, 140.5 (q, J=1.4 Hz), 129.4 (q, J=32.4 Hz), 129.1, 127.6-120.8 (m), 125.3 (q, J=3.7 Hz), 66.6, 52.9, 52.2, 48.3, 41.3.

HRMS (ESI) m/z calcd. for C₁₆H₁₆O₄F3 ([M+H]⁺) 329.1001, found 329.0994.

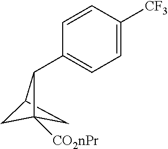

(±)-Propyl 2-(4-(trifluoromethyl)phenyl)bicyclo[1.1.1]pentane-1-carboxylate. Prepared according to Scheme 5 by slowly adding a solution of 1-bromo-4-(trifluoromethyl)benzene (113 mg, 0.50 mmol) and silane in PhCF₃ via syringe pump to a solution of propyl 2-bromobicyclo[1.1.1]pentane-1-carboxylate (233 mg, 1.00 mmol, 2.0 equiv), cesium carbonate, Ir(dF(CF₃))₂(dtbpy)PF₆ and 5% Ni(dtbbpy)Br₂ in PhCF₃. Purification via flash column chromatography (10-75% CH₂Cl₂/hexane) gave 122.1 mg (409 mmol, 82%) as a colorless oil.

¹H NMR (500 MHz, CDCl₃) δ 7.56 (d, J=8.0 Hz, 2H), 7.38 (d, J=8.0 Hz, 2H), 4.08 (t, J=6.7 Hz, 2H), 3.75 (d, J=6.8 Hz, 1H), 2.81 (s, 1H), 2.35 (dd, J=9.7, 3.0 Hz, 1H), 2.19 (d, J=1.9 Hz, 1H), 2.17-2.11 (m, 2H), 1.67 (h, J=7.2 Hz, 2H), 0.94 (t, J=7.4 Hz, 3H).

¹³C NMR (126 MHz, CDCl₃) δ 169.3, 142.9 (q, J=1.4 Hz), 129.0, 128.8 (q, J=32.3 Hz), 125.1 (q, J=3.8 Hz), 124.4 (q, J=271.9 Hz), 66.4, 64.7, 49.9, 47.9, 46.0, 31.1, 22.1, 10.5.

HRMS (ESI) m/z calcd. for C₁₆H₁₆O₄F₃ ([M+H]⁺) 299.1259, found 299.1255.

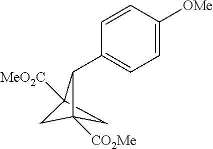 [SMILES: COC(=O)C12CC(C(=O)OC)(C1)C2c1ccc(OC)cc1]

Dimethyl 2-(4-methoxyphenyl)bicyclo[1.1.1]pentane-1,3-dicarboxylate. Prepared according to Scheme 5 by slowly adding a solution of 1-bromo-4-methoxy-benzene (93.5 mg, 0.50 mmol) and silane in PhCF₃ via syringe pump to a solution of dimethyl 2-bromobicyclo[1.1.1]pentane-1,3-dicarboxylate (263 mg, 1.00 mmol, 2.0 equiv), cesium carbonate, Ir(dF(CF₃))₂(dtbpy)PF₆ and 10% Ni(dtbbpy)Br₂ in PhCF₃. Purification via flash column chromatography (0-80% CH₂Cl₂/hexane) gave 110.8 mg (382 mmol, 76%) as a white solid.

¹H NMR (500 MHz, CDCl₃) δ 7.23-7.12 (m, 2H), 6.93-6.78 (m, 2H), 3.98 (d, J=6.6 Hz, 1H), 3.79 (s, 3H), 3.73 (s, 6H), 2.72 (dd, J=10.0, 2.9 Hz, 1H), 2.42-2.31 (m, 3H).

¹³C NMR (101 MHz, CDCl₃) δ 169.6, 158.7, 129.7, 128.6, 113.9, 66.9, 55.4, 52.8, 52.0, 48.0, 41.2.

HRMS (ESI) m/z calcd. for C₁₆H₁₈NO₅Na ([M+H]⁺) 313.1052, found 313.1044.

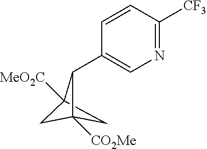 [SMILES: COC(=O)C12CC(C(=O)OC)(C1)C2c1ccc(C(F)(F)F)nc1]

Dimethyl 2-(6-(trifluoromethyl)pyridin-3-yl)bicyclo[1.1.1]pentane-1,3-dicarboxylate. Prepared according to Scheme 5 by slowly adding a solution of 5-bromo-2-(trifluoromethyl)pyridine (113 mg, 0.50 mmol) and silane in PhCF₃ via syringe pump to a solution of dimethyl 2-bromobicyclo[1.1.1]pentane-1,3-dicarboxylate (263 mg, 1.00 mmol, 2.0 equiv), cesium carbonate, Ir(dF(CF₃))₂(dtbpy)PF₆ and 5% Ni(dtbbpy)Br₂ in PhCF₃. Purification via flash column chromatography (5-50% EtOAc/hexane) gave 121.6 mg (369 mmol, 74%) as a white solid.

¹ H NMR (500 MHz, CDCl₃) δ 8.70-8.69 (m, 1H), 7.91 (ddt, J=8.1, 2.2, 0.8 Hz, 1H), 7.65 (dd, J=8.1, 0.9 Hz, 1H), 4.00 (d, J=6.6 Hz, 1H), 3.74 (s, 6H), 2.63 (dd, J=9.6, 3.3 Hz, 1H), 2.50-2.40 (m, 3H).

¹³C NMR (101 MHz, CDCl₃) δ 168.7, 150.4, 146.9 (q, J=34.6 Hz), 138.0, 135.5, 121.7 (q, J=273.9 Hz), 120.1 (q, J=2.7 Hz), 63.9, 53.0, 52.4, 48.8, 41.2.

HRMS (ESI) m/z calcd. for C₁₅H₁₅NO₄F₃ ([M+H]⁺) 330.0953, found 330.0948.

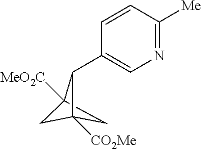 [SMILES: COC(=O)C12CC(C(=O)OC)(C1)C2c1ccc(C)nc1]

Dimethyl 2-(6-methylpyridin-3-yl)bicyclo[1.1.1]pentane-1,3-dicarboxylate. Prepared according to Scheme 5 by slowly adding a solution of 5-bromo-2-methyl-pyridine (86.0 mg, 0.50 mmol) and silane in PhCF₃ via syringe pump to a solution of dimethyl 2-bromobicyclo[1.1.1]pentane-1,3-dicarboxylate (263 mg, 1.00 mmol, 2.0 equiv), cesium carbonate, Ir(dF(CF₃))₂(dtbpy)PF₆ and 10% Ni(dtbbpy)Br₂ in PhCF₃. Purification via sequential flash column chromatography (wash column: 0-5% MeOH/CH₂Cl₂, concentrate fractions, then 1-100% EtOAc/hexane) gave 121.6 mg (369 mmol, 74%) as a brownish-red solid.

¹H NMR (500 MHz, CDCl₃) δ 8.40 (d, J=2.3 Hz, 1H), 7.53 (dd, J=8.0, 2.4 Hz, 1H), 7.11 (d, J=8.0 Hz, 1H), 3.94 (d, J=6.7 Hz, 1H), 3.73 (s, 5H), 2.67 (dd, J=9.4, 3.1 Hz, 1H), 2.53 (s, 3H), 2.45-2.36 (m, 3H).

¹³ C NMR (126 MHz, CDCl₃) δ 169.1, 157.1, 149.2, 136.8, 128.9, 122.9, 64.5, 53.0, 52.2, 48.3, 41.0, 24.2.

HRMS (ESI) m/z calcd. for C₁₅H₁₈NO₄ ([M+H]⁺) 276.1236, found 276.1232.

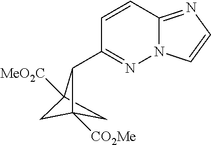 [SMILES: COC(=O)C12CC(C(=O)OC)(C1)C2c1ccc2nccn2n1]

Dimethyl 2-(imidazo[1,2-13]pyridazin-6-yl)bicyclo[1.1.1]pentane-1,3-dicarboxylate. Prepared according to Scheme 5 by slowly adding a solution of 6-bromoimidazo[1,2-b]pyridazine (99.0 mg, 0.50 mmol) and silane in PhCF₃/tBuOH=1/1 via syringe pump to a solution of dimethyl 2-bromobicyclo[1.1.1]pentane-1,3-dicarboxylate (263 mg, 1.00 mmol, 2.0 equiv), cesium carbonate, Ir(dF(CF₃))₂(dtbpy)PF₆ and 10% Ni(dtbbpy)Br₂ in PhCF₃. Purification via flash column chromatography (10-100% MeCN/CH₂Cl₂) gave 63.5 mg (211 mmol, 42%) as a pale yellow crystalline solid.

¹H NMR (500 MHz, CDCl₃) δ 7.89 (dd, J=9.4, 0.7 Hz, 1H), 7.86 (t, J=0.9 Hz, 1H), 7.73 (d, J=1.2 Hz, 1H), 7.14 (d, J=9.3 Hz, 1H), 4.01 (d, J=6.5 Hz, 1H), 3.76 (s, 6H), 2.76 (dd, J=10.3, 3.1 Hz, 1H), 2.48-2.41 (m, 3H).

¹³ C NMR (101 MHz, CDCl₃) δ 168.8, 150.3, 138.3, 133.8, 125.5, 118.5, 117.0, 64.6, 53.3, 52.2, 48.6, 41.3.

HRMS (ESI) m/z calcd. for C₁₅H₁₈NO₄ ([M+H]⁺) 302.1141, found 302.1136.

Temperature control can be important for certain substrates, with substantial drops in yield at higher temperatures. In an embodiment, the temperature of the reaction mixture is kept below 40° C. In another embodiment, the temperature of the reaction mixture is kept below about 30° C., about 20° C., about 10° C., about 0° C., about −10° C., about −20° C., or about −30° C. In other embodiments, the temperature of the reaction mixture is between about −20° C. and about 40° C. In another embodiment, the temperature of the reaction mixture is between about 10° C. and about 30° C. In a preferred embodiment, the temperature of the reaction mixture is about 25° C.

For polar (hetero)aryl halides insoluble in PhCF₃, using a co-solvent in low amounts in the solution can be beneficial. In an embodiment, the co-solvent is a polar solvent. In an embodiment, the co-solvent is a polar protic solvent. In an embodiment, the co-solvent is an alkyl alcohol. In a further embodiment, the co-solvent is methanol, ethanol, n-propyl alcohol, i-propyl alcohol, tent-butyl alcohol, tent-amyl alcohol, or n-amyl alcohol. In a preferred embodiment, the co-solvent is tent-butyl alcohol. Addition of a co-solvent was not deleterious to the yield even in cases that do not necessitate a polar co-solvent. Certain aprotic polar solvents, such as acetone and acetonitrile, were observed to be suitable in low amounts, but deleterious to yield in higher amounts.

In some embodiments, lower nickel catalyst loading is preferable for cross-coupling of electron-deficient (hetero)aryl halides. Without wishing to be bound by any particular theory, lower catalyst loading may reduce (hetero)aryl homocoupling. Examples of electron-deficient (hetero)aryl halides include, but are not limited to, p-trifluoromethyl phenyl halide and p-trifluoromethyl pyridine halide. In an embodiment, the lower nickel catalyst loading is between about 1 mol percent and about 5 mol percent. In a preferred embodiment, the lower nickel catalyst loading is about 5 mol percent.

In some embodiments, higher nickel catalyst loading is preferable for cross-coupling of electron-rich (hetero)aryl halides. Examples of electron-rich (hetero)aryl halides include, but are not limited to, p-anisole halide, p-alkyl phenyl halide, p-alkyl pyridine, and imidazo[1,2-b]pyridazine halide. In an embodiment, the higher nickel catalyst loading is between about 6 mol percent and about 15 mol percent. In a preferred embodiment, the higher nickel catalyst loading is about 10 mol percent.

A number of different nickel catalysts may be employed without an appreciable drop in yield. In some embodiments, nickel salts may be used to form the ligated nickel species in situ. For example, NiBr₂·glyme and dtbpy can be used instead of Ni(dtbbpy)Br₂. In a preferred embodiment, the nickel catalyst is fully dissolved before starting irradiation and addition of (hetero)aryl halide and silane. Dissolution could be achieved, for example, by sonication of the solution containing BCP bromide, cesium carbonate, Ir photocatalyst, and a Ni complex precatalysts, for example, for about 15 minutes.

1,2,3-trisubstituted BCP derivatives can be synthesized according to Scheme 6, wherein R corresponds to R¹¹ as described in any of the embodiments, or aspects thereof, described herein.

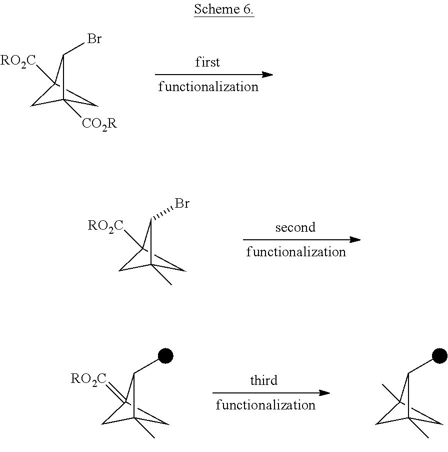

The following 1,2,3-trisubstituted BCP derivatives:

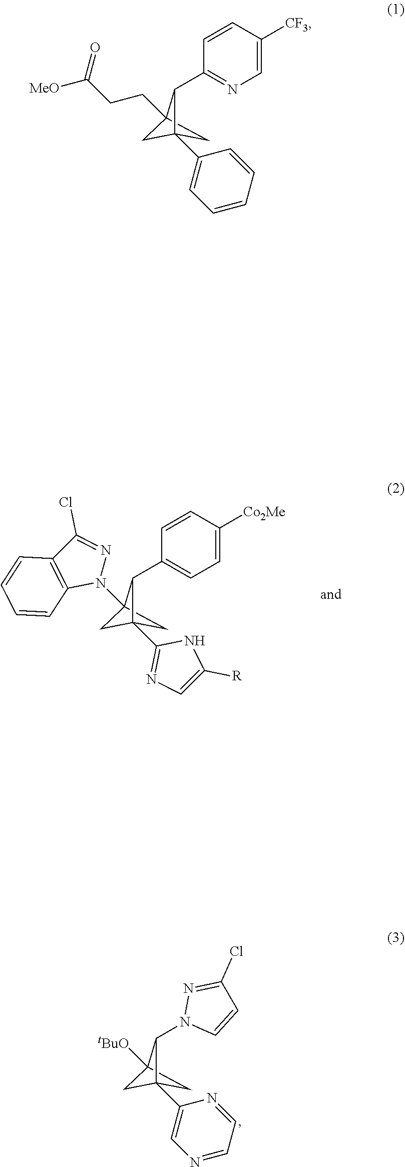

can be synthesized according to Scheme 6 using the following derivatization reactions:

| BCP | First | Second | Third |
|---|---|---|---|
| Derivative | Functionalization | Functionalization | Functionalization |
| 1 | Decarboxylative | Cross electrophile | Decarboxylative |
| | arylation | C—C coupling | alkylation |
| 2 | Decarboxylative | Cross electrophile | Decarboxylative |
| | heteroarylation | C—C coupling | amination |
| 3 | Decarboxylative | sp³ C—N coupling | Decarboxylative |
| | heteroarylation | | etherification |

1,2-disubstituted BCP derivatives can be synthesized according to Scheme 7, wherein R corresponds to R¹¹ as described in any of the embodiments, or aspects thereof, described herein.

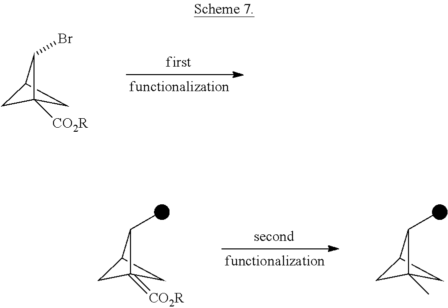

The following 1,2-disubstituted BCP derivatives:

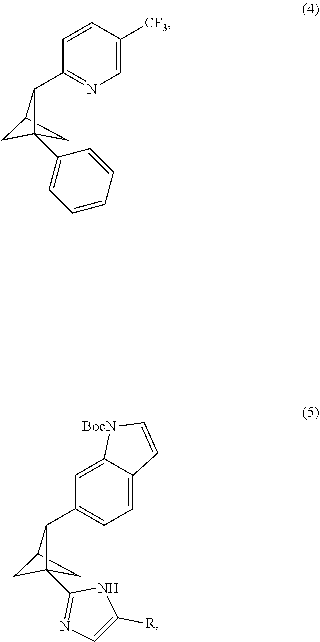

wherein R is any suitable substituent described herein, and

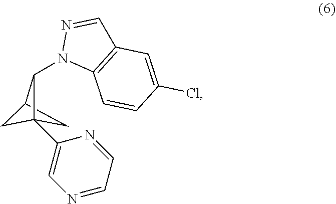 [SMILES: Clc1ccc2c(cnn2C2C3CC2(c2cnccn2)C3)c1]

can be synthesized according to Scheme 7 using the following derivatization reactions:

| BCP | First | Second |
|---|---|---|
| Derivative | Functionalization | Functionalization |
| 4 | Cross electrophile | Decarboxylative |
| | C—C coupling | arylation |
| 5 | Cross electrophile | Decarboxylative |
| | C—C coupling | heteroarylation |
| 6 | sp³ C—N coupling | Decarboxylative |
| | | heteroarylation |

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A compound of the following structural formula: 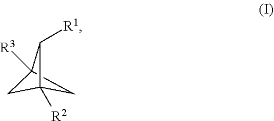
or a salt thereof, wherein:
R¹ is halo, —OH or —NH₂; and
R² and R³ are each independently —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl, (C₅-C₁₅)heteroaryl, —CH₂-halo, —CH₂-OR¹¹, —CH₂—N(R¹¹)R¹², —C(H)(OR¹²)(NR¹¹R¹²) or —C(H)(OR¹¹)(OR¹²);
R¹⁰, for each occurrence, is independently —H, halo, —NR¹¹R¹², —OR¹¹, —O—NR¹¹R¹², (C₁-C₁₀)aliphatic, (C₁-C₁₀)heteroaliphatic, (C₆-C₁₅)aryl or (C₅-C₁₅)heteroaryl; and
R¹¹ and R¹², for each occurrence, are independently H, (C₁-C₁₀)aliphatic, (C₁-C₁₀)heteroaliphatic, (C₆-C₁₅)aryl or (C₅-C₁₅)heteroaryl, or taken together with the atoms to which they are attached and any intervening atoms, form a 3-15-membered cyclyl,
wherein each aliphatic, heteroaliphatic, aryl, heteroaryl and cyclyl is optionally and independently substituted,
provided the compound is not 2-chlorobicyclo[1.1.1]pentane, 1,2-dichlorobicylo[1.1.1]pentane, 2-chlorobicyclo[1.1.1]pentane-1,3-dicarboxylic acid, dimethyl 2-chlorobicyclo[1.1.1]pentane-1,3-dicarboxylate, 2-chloro-3-methoxycarbonylbicyclo[1.1.1]pentane-1-carboxylic acid, methyl 2-chloro-3-hydroxymethylbicyclo[1.1.1]pentane-1-carboxylate, methyl 2-chloro-3-formylbicyclo[1.1.1]pentane-1-carboxylate, 2-hydroxybicyclo[1.1.1]pentane, bicyclo[1.1.1]pentan-2-amine or dimethyl 2-fluorobicyclo[1.1.1]pentane-1,3-dicarboxylate, or a salt of any of the foregoing.

2. The compound of claim 1, wherein R¹ is bromo, iodo, —OH or —NH₂.

3. The compound of claim 1 or claim 2, wherein R¹ is bromo or iodo.

4. The compound of any one of claims 1-3, wherein R¹ is bromo.

5. The compound of any one of claims 1-4, wherein R² and R³ are the same.

6. The compound of any one of claims 1-4, wherein R² and R³ are different.

7. The compound of any one of claims 1-6, wherein:
R² is —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²); and
R³ is —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²).

8. The compound of any one of claims 1-6, wherein R² and R³ are each independently —C(O)R¹⁰ or —H.

9. The compound of any one of claims 1-8, wherein R¹⁰, for each occurrence, is independently —OR¹¹ or —O—NR¹¹R¹².

10. The compound of any one of claims 1-9, wherein R¹⁰, for each occurrence, is independently —OR¹¹.

11. The compound of claim 10, wherein R¹¹, for each occurrence, is —H or (C₁-C₁₀)alkyl.

12. The compound of claim 11, wherein R¹¹, for each occurrence, is —H.

13. The compound of any one of claims 1-9, wherein —O—NR¹¹R¹² is —O—N-phthalimidyl or —O—N-(2-thiopyridonyl), each of which is optionally and independently substituted.

14. The compound of any one of claims 1-10, wherein R¹¹ and R¹², for each occurrence, are independently H, (C₁-C₁₀)aliphatic, (C₁-C₁₀)heteroaliphatic, (C₆-C₁₅)aryl or (C₅-C₁₅)heteroaryl.

15. The compound of any one of claims 1-5, represented by the following structural formula: 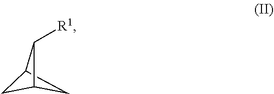
or a salt thereof.

16. The compound of any one of claims 1-4, 6 and 9-14, represented by the following structural formula: 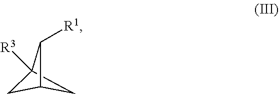
or a salt thereof, wherein R³ is —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²).

17. The compound of claim 16, wherein R³ is —C(O)R¹⁰.

18. The compound of any one of claims 1-7 and 9-14, wherein R² and R³ are each independently —C(O)R¹⁰, —S(O)₂R¹⁰, —N(R¹¹)C(O)R¹⁰, —OC(O)R¹⁰, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²).

19. The compound of claim 18, wherein R² and R³ are each independently —C(O)R¹⁰.

20. A method of making a compound of the following structural formula: 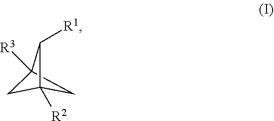
or a salt thereof, wherein:
R¹ is halo, —OH or —NH₂; and
R² and R³ are each independently —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²);
R¹⁰, for each occurrence, is independently —H, halo, —NR¹¹R¹², —OR¹¹, —O—NR¹¹R¹², (C₁-C₁₀)aliphatic, (C₁-C₁₀)heteroaliphatic, (C₆-C₁₅)aryl or (C₅-C₁₅)heteroaryl; and
R¹¹ and R¹², for each occurrence, are independently H, (C₁-C₁₀)aliphatic, (C₁-C₁₀)heteroaliphatic, (C₆-C₁₅)aryl or (C₅-C₁₅)heteroaryl, or taken together with the atoms to which they are attached and any intervening atoms, form a 3-15-membered cyclyl,
wherein each aliphatic, heteroaliphatic, aryl, heteroaryl and cyclyl is optionally and independently substituted,
the method comprising reacting a compound of the following structural formula: 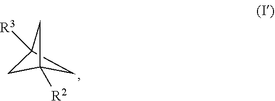
or a salt thereof, wherein R² and R³ are as described for the compound of structural formula (I), with a hydrogen atom abstractor and an R l source, thereby making the compound of structural formula I, or a salt thereof.

21. The method of claim 20, wherein the hydrogen atom abstractor is photoreactive.

22. The method of claim 20 or 21, wherein the hydrogen atom abstractor is an aromatic ketone, decatungstate anion or uranyl cation.

23. The method of any one of claims 20-22, wherein the hydrogen atom abstractor is a N-chloro compound.

24. The method of claim 23, wherein the N-chloro compound is N-chlorosuccinimide, 1,3-dichloro-5,5-dimethylimidazolidine-2,4-dione, 2-chloroisoindoline-1,3-dione, 1,3,5-trichloro-1,3,5-triazinane-2,4,6-trione, N-chloro-N-(phenyl sulfonyl)benzenesulfonamide or N-chloro 4-methylbenzenesulfonamide.

25. The method of any one of claims 20-24, wherein the R¹ source is a halogenating agent.

26. The method of claim 25, wherein the halogenating agent is a brominating agent.

27. The method of claim 26, wherein the brominating agent is bromotrichloromethane, tetrabromomethane, N-bromosuccinimide, N-bromophthalimide, 1,3-dibromo-5,5-dimethylimidazolidine-2,4-dione or 2-bromobenzo[d]isothiazol-3(2H)-one 1,1-dioxide.

28. The method of claim 25, wherein the halogenating agent is an iodinating agent.

29. The method of claim 28, wherein the iodinating agent is a perfluoroalkyliodide, diiodomethane or N-iodosuccinimide.

30. The method of claim 25, wherein the halogenating agent is a chlorinating agent.

31. The method of claim 30, wherein the chlorinating agent is N-chlorosuccinimide, 1,3-dichloro-5,5-dimethylimidazolidine-2,4-dione, 2-chloroisoindoline-1,3-dione, 1,3,5-trichloro-1,3,5-triazinane-2,4,6-trione, N-chloro-N-(phenyl sulfonyl)benzenesulfonamide or N-chloro 4-methylbenzenesulfonamide.

32. The method of any one of claims 20-31, comprising reacting the compound of structural formula I′, or a salt thereof, with the hydrogen atom abstractor and the R¹ source in a solvent.

33. The method of claim 32, wherein the solvent is an organic solvent, water or a combination of an organic solvent and water.

34. The method of any one of claims 20-33, wherein reacting includes irradiating the hydrogen atom abstractor in the presence of the compound of structural formula I′, or a salt thereof, and the R¹ source.

35. The method of claim 34, wherein irradiating comprises exposing to light having a wavelength of from about 100 nm to about 525 nm.

36. The method of claim 35, wherein the light has a wavelength of from about 365 nm to about 500 nm.

37. The method of any one of claims 20-36, wherein R² is H and R³ is CO₂H, or R² and R³ are each CO₂H.

38. The method of claim 37, further comprising esterifying the carboxylic acid of R^{2 ,} R³ or R² and R³.

39. A method of derivatizing a compound of the following structural formula: 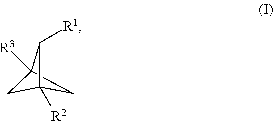
or a salt thereof, wherein:
R¹ is halo, —OH or —NH₂; and
R² and R³ are each independently —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²);
R¹⁰, for each occurrence, is independently —H, halo, —NR¹¹R¹², —OR¹¹, —O—NR¹¹R¹², (C₁-C₁₀)aliphatic, (C₁-C₁₀)heteroaliphatic, (C₆-C₁₅)aryl or (C₅-C₁₅)heteroaryl; and
R¹¹ and R¹², for each occurrence, are independently H, (C₁-C₁₀)aliphatic, (C₁-C₁₀)heteroaliphatic, (C₆-C₁₅)aryl or (C₅-C₁₅)heteroaryl, or taken together with the atoms to which they are attached and any intervening atoms, form a 3-15-membered cyclyl,
wherein each aliphatic, heteroaliphatic, aryl, heteroaryl and cyclyl is optionally and independently substituted,
the method comprising subjecting the compound of structural formula I, or a suitably protected form thereof, or a salt of either of the foregoing, to a derivatization reaction, thereby derivatizing the compound of structural formula I, or a salt thereof

40. A method of making a compound of the following structural formula: 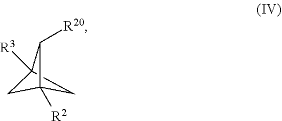
or a salt thereof, wherein:
R²⁰ is (C₆-C₁₅)aryl or (C₅-C₁₅)heteroaryl;
R² and R³ are each independently —C(O)R¹⁰, —S(O)₂R¹⁰, —N(R¹¹)C(O)R¹⁰, —OC(O)R¹⁰, —H, halo, —CN, —OR¹¹, —N(R¹¹)R¹², (C₆-C₁₅)aryl, (C₅-C₁₅)heteroaryl, —CH₂-halo, —CH₂—OR¹¹, —CH₂—N(R¹¹)R¹², —C(H)(OR¹²)(NR¹¹R¹²) or —C(H)(OR¹¹)(OR¹²);
R¹⁰, for each occurrence, is independently —H, halo, —NR¹¹R¹², —OR¹¹, —O—NR¹¹R¹², (C₁-C₁₀)aliphatic, (C₁-C₁₀)heteroaliphatic, (C₆-C₁₅)aryl or (C₅-C₁₅)heteroaryl; and
R¹¹ and R¹², for each occurrence, are independently H, (C₁-C₁₀)aliphatic, (C₁-C₁₀)heteroaliphatic, (C₆-C₁₅)aryl or (C₅-C₁₅)heteroaryl, or taken together with the atoms to which they are attached and any intervening atoms, form a 3-15-membered cyclyl,
wherein each aliphatic, heteroaliphatic, aryl, heteroaryl and cyclyl is optionally and independently substituted,
the method comprising reacting a compound of structural formula (I): 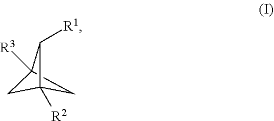
or a salt thereof, wherein R¹ is halo, —OH or -—H₂; and R² and R³ are as described for the compound of structural formula (IV),
with an R¹ activator, a photocatalyst and R²⁰-M in a solvent, wherein M is a transition metal, thereby making a compound of structural formula (IV), or a salt thereof.

41. The method of claim 40, wherein the (C₆-C₁₅)aryl or (C₅-C₁₅)heteroaryl of R²⁰ is optionally and independently substituted with one or more substituents selected from halo, halo(C₁-C₁₀)alkyl, (C₁-C₁₀)alkyl, —O-(C₁-C₁₀)alkyl, —O-halo(C₁-C₁₀)alkyl or —C(O)R¹³, wherein R¹³, for each occurrence, is independently —OH, —O-(C₁-C₁₀)alkyl or —O-halo(C₁-C₁₀)alkyl.

42. The method of claim 40 or 41, wherein R¹ is chloro, bromo or iodo.

43. The method of claim 42, wherein R¹ is bromo.

44. The method of claim 42 or 43, wherein the R¹ activator is a silane.

45. The method of claim 44, wherein the silane is tris(trimethylsilyl)silane, tris(trimethylsilyl)silanol, N-tris(trimethylsilyl)silyladamantan-1-amine, N-tris(trimethylsilyl)silyl-t-butylamine or N-methyl-N-tris(trimethylsilyl)silyl-t-butylamine.

46. The method of claim 40 or 41, wherein R¹ is —OH.

47. The method of claim 46, wherein the R¹ activator is an N-heterocyclic carbene.

48. The method of claim 40 or 41, wherein R¹ is —NH₂.

49. The method of claim 48, wherein the R¹ activator is an electron-rich aldehyde or a Katritzky salt.

50. The method of any one of claims 40-49, wherein M is nickel or copper.

51. The method of any one of claims 40-50, wherein the photocatalyst is an iridium-based photocatalyst.

52. The method of any one of claims 40-51, wherein the solvent is an organic solvent, water, or a combination of an organic solvent and water.

53. The method of any one of claims 40-52, wherein reacting comprises irradiating the compound of formula (I), or a salt thereof, R¹ activator, photocatalyst and R²⁰-M in the solvent.

54. The method of claim 53, wherein irradiating comprises exposing to light having a wavelength of from about 100 nm to about 525 nm.

55. The method of claim 54, wherein the light has a wavelength of from about 365 nm to about 500 nm.

56. The method of any one of claims 40-55, comprising reacting the compound of structural formula (I), or a salt thereof, R¹ activator, photocatalyst and R²⁰-M in the further presence of a base.

57. The method of claim 56, wherein the base is an inorganic base.

58. The method of claim 56, wherein the base is an organic amine base.

59. The method of claim 56, wherein the base is a superbase.

60. The method of any one of claims 40-59, wherein the method results in formation of a covalent carbon-carbon bond between 2-carbon of the compound of structural formula (I), or a salt thereof, and R²⁰.

61. The method of claim 60, further comprising contacting R²⁰—X, wherein X is halo and is attached to a ring carbon of R²⁰, with a transition metal salt to form R²⁰-M.

62. The method of claim 60 or 61, further comprising contacting R²⁰—X, wherein X is halo and is attached to a ring carbon of R²⁰, with a transition metal complex to form R²⁰-M.

63. The method of any one of claims 40-62, wherein R²⁰ is (C₅-C₁₅)heteroaryl containing at least one ring nitrogen, and the method results in formation of a covalent bond between 2-carbon of the compound of structural formula (I), or a salt thereof, and one ring nitrogen of the (C₅-C₁₅)heteroaryl of R²⁰.

64. The method of claim 63, further comprising contacting R²⁰—H with a transition metal salt to form R²⁰-M.

65. The method of claim 63 or 64, further comprising contacting R²⁰—H with a transition metal complex to form R²⁰-M.

66. The method of claim 61 or 64, wherein the transition metal salt is a nickel salt or copper salt.

67. The method of claim 62 or 65, wherein the transition metal complex is a nickel complex or copper complex.

68. The method of any one of claims 40-67, wherein:
R² and R³ are each independently —C(O)R¹⁰ or —H;
R¹⁰, for each occurrence, is independently —OR¹¹ or —O—NR¹¹R¹²; and
R¹¹ and R¹², for each occurrence, are independently H or (C₁-C₁₀)alkyl, or taken together with the atoms to which they are attached and any intervening atoms, form a 3-15-membered cyclyl, which is optionally and independently substituted.

69. The method of claim 68, wherein —O—NR¹¹R¹² is —O—N-phthalimidyl or —O—-N-(2-thiopyridonyl), each of which is optionally and independently substituted.

70. The method of claim 68 or 69, further comprising performing a decarboxylative cross-coupling on the compound of structural formula (I) or (IV), or a salt thereof, or (I) and (IV), or a salt of any of the foregoing.
